SEQUENCE LISTING (50 sequences)

SEQ ID NO: 1 - DNA, 31 bp, Artificial sequence
  Primer
gctagcagccaccatgcgaccctccgggacg

SEQ ID NO: 2 - DNA, 27 bp, Artificial sequence
  Primer
gcgccacatcgttcggaaggactcgag

SEQ ID NO: 3 - DNA, 22 bp, Artificial sequence
  Primer
caagctctgtccgtatcgatca

SEQ ID NO: 4 - DNA, 25 bp, Artificial sequence
  Primer
tggacgataatcggaaaagtaatca

SEQ ID NO: 5 - DNA, 27 bp, Artificial sequence
  Probe
ctggatcgtgaacagttcaaccccgat

SEQ ID NO: 6 - DNA, 21 bp, Artificial sequence
  Primer
aaagcaaggtctccccacaag

SEQ ID NO: 7 - DNA, 24 bp, Artificial sequence
  Primer
tgaagggtctgtgctagatcaaaa

SEQ ID NO: 8 - DNA, 19 bp, Artificial sequence
  Probe
tgccacatcgccaccccgt

SEQ ID NO: 9 - DNA, 20 bp, Artificial sequence
  Synthetic oligonucleotide
atccctttcttccgcatgtg

SEQ ID NO: 10 - DNA, 20 bp, Artificial sequence
  Synthetic oligonucleotide
ctgctagcctctggatttga

SEQ ID NO: 11 - DNA, 20 bp, Artificial sequence
  Synthetic oligonucleotide
agcgcagacaaacccatcac

SEQ ID NO: 12 - DNA, 20 bp, Artificial sequence
  Synthetic oligonucleotide
ggcatatgcagataatgttc

SEQ ID NO: 13 - DNA, 20 bp, Artificial sequence
  Synthetic oligonucleotide
  misc_feature (1)..(9): bases at these positions are RNA
  misc_feature (11)..(11): bases at these positions are RNA
  misc_feature (13)..(20): bases at these positions are RNA
cugcuagcctctggauuuga

SEQ ID NO: 14 - DNA, 20 bp, Artificial sequence
  Synthetic oligonucleotide
  misc_feature (1)..(1): bases at these positions are RNA
  misc_feature (3)..(4): bases at these positions are RNA
  misc_feature (6)..(9): bases at these positions are RNA
  misc_feature (11)..(15): bases at these positions are RNA
  misc_feature (19)..(20): bases at these positions are RNA
ctgctagcctcuggatttga

SEQ ID NO: 15 - DNA, 16 bp, Artificial sequence
  Synthetic oligonucleotide
ggctactacgccgtca

SEQ ID NO: 16 - DNA, 16 bp, Artificial sequence
  Synthetic oligonucleotide
gcatgttctcacatta

SEQ ID NO: 17 - DNA, 16 bp, Artificial sequence
  Synthetic oligonucleotide
ggtcgaccgaagtcat

SEQ ID NO: 18 - DNA, 20 bp, Artificial sequence
  Synthetic oligonucleotide
ccttccctgaaggttcctcc

SEQ ID NO: 19 - DNA, 17 bp, Artificial sequence
  Synthetic oligonucleotide
aaaaaaaaaaaaaaaaa

SEQ ID NO: 20 - DNA, 16 bp, Artificial sequence
  Synthetic oligonucleotide
cccccccccccccccc

SEQ ID NO: 21 - DNA, 16 bp, Artificial sequence
  Synthetic oligonucleotide
tttttttttttttttt

SEQ ID NO: 22 - DNA, 20 bp, Artificial sequence
  Synthetic oligonucleotide
aggaaggaagctggcgatct

SEQ ID NO: 23 - DNA, 20 bp, Artificial sequence
  Synthetic oligonucleotide
ctaacatgcaatactgcaga

SEQ ID NO: 24 - DNA, 20 bp, Artificial sequence
  Synthetic oligonucleotide
cagcaggcaactgtcgctga

SEQ ID NO: 25 - DNA, 20 bp, Artificial sequence
  Synthetic oligonucleotide
ttttggcaaagtaatcgtcc

SEQ ID NO: 26 - DNA, 20 bp, Artificial sequence
  Synthetic oligonucleotide
acacggtattgcccttgaaa

SEQ ID NO: 27 - DNA, 20 bp, Artificial sequence
  Synthetic oligonucleotide
taagtactactgggccatgg

SEQ ID NO: 28 - DNA, 20 bp, Artificial sequence
  Synthetic oligonucleotide
tggtaatccactttcagagg

SEQ ID NO: 29 - DNA, 20 bp, Artificial sequence
  Synthetic oligonucleotide
acccaattcagaaggaagga

SEQ ID NO: 30 - DNA, 16 bp, Artificial sequence
  Synthetic oligonucleotide
ttctgggcagccccac

SEQ ID NO: 31 - DNA, 16 bp, Artificial sequence
  Synthetic oligonucleotide
ggccatcacgccacag

SEQ ID NO: 32 - DNA, 16 bp, Artificial sequence
  Synthetic oligonucleotide
ctttccagaggggcca

SEQ ID NO: 33 - DNA, 16 bp, Artificial sequence
  Synthetic oligonucleotide
tccacagtcttctggg

SEQ ID NO: 34 - DNA, 16 bp, Artificial sequence
  Synthetic oligonucleotide
tggcagtgatggcatg

SEQ ID NO: 35 - DNA, 16 bp, Artificial sequence
  Synthetic oligonucleotide
gactgtggtcatgagc

SEQ ID NO: 36 - DNA, 16 bp, Artificial sequence
  Synthetic oligonucleotide
ccttccacaatgccaa

SEQ ID NO: 37 - DNA, 16 bp, Artificial sequence
  Synthetic oligonucleotide
agttgtcatggatgac

SEQ ID NO: 38 - DNA, 16 bp, Artificial sequence
  Synthetic oligonucleotide
aagcagttggtggtgc

SEQ ID NO: 39 - DNA, 16 bp, Artificial sequence
  Synthetic oligonucleotide
aggatgcattgctgac

SEQ ID NO: 40 - DNA, 20 bp, Artificial sequence
  Synthetic oligonucleotide
tcgtctgtgcatctctcctg

SEQ ID NO: 41 - DNA, 20 bp, Artificial sequence
  Synthetic oligonucleotide
gagtcagtatcccagtgtct

SEQ ID NO: 42 - DNA, 20 bp, Artificial sequence
  Synthetic oligonucleotide
aatctccttgctgtatttgt

SEQ ID NO: 43 - DNA, 20 bp, Artificial sequence
  Synthetic oligonucleotide
ctgatgatctgcaggttttc

SEQ ID NO: 44 - DNA, 20 bp, Artificial sequence
  Synthetic oligonucleotide
aacgaggtactgtgtaagtc

SEQ ID NO: 45 - DNA, 20 bp, Artificial sequence
  Synthetic oligonucleotide
  misc_feature (1)..(6): bases at these positions are RNA
  misc_feature (9)..(20): bases at these positions are RNA
auaaucttccagggccacaa

SEQ ID NO: 46 - DNA, 20 bp, Artificial sequence
  Synthetic oligonucleotide
  misc_feature (1)..(9): bases at these positions are RNA
  misc_feature (11)..(11): bases at these positions are RNA
  misc_feature (16)..(20): bases at these positions are RNA
cuguugggatattttagccu

SEQ ID NO: 47 - DNA, 20 bp, Artificial sequence
  Synthetic oligonucleotide
atattgcatcagatctcaat

SEQ ID NO: 48 - DNA, 20 bp, Artificial sequence
  Synthetic oligonucleotide
cgtcgtcgtcatcctcgtcc

SEQ ID NO: 49 - DNA, 20 bp, Artificial sequence
  Synthetic oligonucleotide
agtctagggtcacaatctgg

SEQ ID NO: 50 - DNA, 20 bp, Artificial sequence
  Synthetic oligonucleotide
ccaggctggttatgactcag

(19) United States
(12) Patent Application Publication
(10) Pub. No.: US 2020/0407717 A1
(43) Pub. Date: Dec. 31, 2020

(54) METHODS OF MODULATING ANTISENSE ACTIVITY

(71) Applicant: Ionis Pharmaceuticals, Inc., Carlsbad, CA (US)

(72) Inventors: Shiyu Wang, Encinitas, CA (US); Alexey Revenko, San Diego, CA (US); Xue-hai Liang, Del Mar, CA (US); Stanley T. Crooke, Carlsbad, CA (US)

(73) Assignee: Ionis Pharmaceuticals, Inc., Carlsbad, CA (US)

(21) Appl. No.: 16/968,490

(22) PCT Filed: Feb. 13, 2019
(86) PCT No.: PCT/US2019/017836

§ 371 Date: Aug. 7, 2020

Related U.S. Application Data
(60) Provisional application No. 62/630,633, filed on Feb. 14, 2018.

(51) Int. Cl. C12N 15/11; A61K 31/7105; A61K 45/06

(52) U.S. Cl. CPC C12N 15/111; A61K 31/7105; A61K 45/06; C12N 2320/31; C12N 2310/341; C12N 2310/315; C12N 2310/11

(57) ABSTRACT

Disclosed herein are methods for increasing antisense activity by modulating EGFR. In certain embodiments, a compound comprising an antisense oligonucleotide is co-administered with an EGFR modulator.

### SEQUENCE LISTING

The present application is being filed along with a Sequence Listing in electronic format. The Sequence Listing is provided as a file entitled CORE0147WOSEQ_ST25.txt, created Jan. 31, 2019, which is 12 Kb in size. The information in the electronic format of the sequence listing is incorporated herein by reference in its entirety.

### BACKGROUND

Epidermal growth factor receptor (EGFR) is a receptor tyrosine kinase with a large extracellular region, a single transmembrane domain, an intracellular juxtamembrane region, and a cytoplasmic domain. The extracellular region of EGFR contains two homologous ligand binding domains, and the cytoplasmic region contains the tyrosine kinase domain and a C-terminal regulatory doman. Binding of EGF to the extracellular region triggers tyrosine phosphorylation of the cytoplasmic domain, which initiates EGFR endocytosis and degradation. EGFR is highly expressed in carcinomas and selected cancer cell lines such as A431 cells. In these carcinoma cells, EGFR is constitutively internalized and mediates a series of signaling cascades that are required for the survival of carcinoma cells.

The mechanisms by which antisense compounds, including antisense oligonucleotides, are taken up into cells in the absence of transfection reagents or uptake-enhancing conjugate groups are not fully understood. Internalization of antisense compounds, such as antisense oligonucleotides, occurs through endocytic pathways, and the uptake pathways resulting in pharmacological effects are referred to as productive uptake pathways.

### SUMMARY OF THE INVENTION

The present disclosure provides methods of increasing antisense activity by modulating EGFR. The methods provided herein comprise contacting a cell with an antisense compound and contacting a cell with an EGFR modulator. In certain embodiments, the EGFR modulation is modulation of EGFR trafficking, signaling, internalization, and/or expression. In certain embodiments, the antisense activity of the antisense compound is reduction of the level of a target nucleic acid. In certain embodiments, the antisense activity of the antisense compound is splicing modulation of a target nucleic acid. In certain embodiments, the antisense activity of the antisense compound is increase of the level of a target nucleic acid. In certain embodiments, the methods herein comprising EGFR modulation result in a level of antisense activity that is greater than the level of antisense activity that occurs when EGFR is not modulated.

### BRIEF DESCRIPTION OF FIGURES

FIG. 1 shows western blots probed for total EGFR, Ku80, La, CD44, and/or TCP1β, as indicated on the left of each blot.

FIG. 2 shows western blots probed for total EGFR, TCP1β and CD44.

FIG. 3 shows a silver stained SDS-PAGE gel above and a western blot for EGFR below.

FIG. 4 is a western blot probed for total EGFR, phosphorylated EGFR, nucleolin, and TCP1β.

FIG. 5 is a western blot probed for total EGFR, nucleolin, and TCP1β.

FIG. 6 shows western blots for EGFR, s100a10, phosphorylated EGFR, total EGFR, phosphorylated ERK, and total ERK.

### DETAILED DESCRIPTION OF THE INVENTION

Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described.

### Definitions

As used herein, “2′-deoxynucleoside” means a nucleoside comprising 2′-H(H) ribosyl sugar moiety, as found in naturally occurring deoxyribonucleic acids (DNA). In certain embodiments, a 2′-deoxynucleoside may comprise a modified nucleobase or may comprise an RNA nucleobase (uracil).

As used herein, “2′-fluoro” or “2′-F” means a 2′-F in place of the 2′-OH group of a ribosyl ring of a sugar moiety.

As used herein, “2′-substituted nucleoside” or “2-modified nucleoside” means a nucleoside comprising a 2′-substituted or 2′-modified sugar moiety. As used herein, “2′-substituted” or “2-modified” in reference to a sugar moiety means a sugar moiety comprising at least one 2′-substituent group other than H or OH.

As used herein, “antisense activity” means any detectable and/or measurable change attributable to the hybridization of an antisense compound to its target nucleic acid. In certain embodiments, antisense activity is a decrease in the amount or expression of a target nucleic acid compared to target nucleic acid levels in the absence of the antisense compound.

As used herein, “antisense compound” means a compound comprising an antisense oligonucleotide and optionally one or more additional features, such as a conjugate group or terminal group.

As used herein, “antisense oligonucleotide” means an oligonucleotide having a nucleobase sequence that is at least partially complementary to a target nucleic acid.

As used herein, “ameliorate” in reference to a method means improvement in at least one symptom and/or measurable outcome relative to the same symptom or measurable outcome in the absence of or prior to performing the method. In certain embodiments, amelioration is the reduction in the severity or frequency of a symptom or the delayed onset or slowing of progression in the severity or frequency of a symptom and/or disease.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cEt” or “constrained ethyl” means a ribosyl bicyclic sugar moiety wherein the second ring of the bicyclic sugar is formed via a bridge connecting the 4′-carbon and the 2′-carbon, wherein the bridge has the formula 4′-CH(CH₃)—O-2′, and wherein the methyl group of the bridge is in the S configuration.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms that is directly or indirectly attached to an oligonucleotide. Conjugate groups include a conjugate moiety and a conjugate linker that attaches the conjugate moiety to the oligonucleotide.

As used herein, “conjugate linker” means a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “fully modified” in reference to a modified oligonucleotide means a modified oligonucleotide in which each sugar moiety is modified. “Uniformly modified” in reference to a modified oligonucleotide means a fully modified oligonucleotide in which each sugar moiety is the same. For example, the nucleosides of a uniformly modified oligonucleotide can each have a 2′-MOE modification but different nucleobase modifications, and the internucleoside linkages may be different.

As used herein, “gapmer” means an antisense oligonucleotide comprising an internal “gap” region having a plurality of nucleosides that support RNase H cleavage positioned between external “wing” regions having one or more nucleosides, wherein the nucleosides comprising the internal gap region are chemically distinct from the terminal wing nucleosides of the external wing regions.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting” or “inhibition” in refers to a partial or complete reduction. For example, inhibiting the expression of a target nucleic acid means a partial or complete reduction of expression of the nucleic acid, e.g., a reduction in the amount of protein produced from the target nucleic acid, and does not necessarily indicate a total elimination of the protein or target nucleic acid.

As used herein, the terms “internucleoside linkage” means a group or bond that forms a covalent linkage between adjacent nucleosides in an oligonucleotide. As used herein “modified internucleoside linkage” means any internucleoside linkage other than a naturally occurring, phosphate internucleoside linkage. Non-phosphate linkages are referred to herein as modified internucleoside linkages. “Phosphorothioate linkage” means a modified phosphate linkage in which one of the non-bridging oxygen atoms is replaced with a sulfur atom. A phosphorothioate internucleoside linkage is a modified internucleoside linkage. Modified internucleoside linkages include linkages that comprise abasic nucleosides. As used herein, “abasic nucleoside” means a sugar moiety in an oligonucleotide that is not directly connected to a nucleobase. In certain embodiments, an abasic nucleoside is adjacent to one or two nucleosides in an oligonucleotide.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar” or “non-bicyclic modified sugar moiety” means a modified sugar moiety that comprises a modification, such as a substitutent, that does not form a bridge between two atoms of the sugar to form a second ring.

As used herein, “linked nucleosides” are nucleosides that are connected in a continuous sequence (i.e. no additional nucleosides are present between those that are linked).

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulation” means a perturbation of function, formation, activity, size, amount, trafficking, and/or localization. As used herein, an “EGFR modulator” is a compound or composition that modulates EGFR function, formation, activity (e.g., signaling), size, amount, trafficking (e.g., internalization), and/or localization.

As used herein, “MOE” means methoxyethyl. “2′-MOE” means a 2′-OCH₂CH₂OCH₃ group in place of the 2′-OH group of a ribosyl ring of a sugar moiety.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means a naturally occurring nucleobase or a modified nucleobase. As used herein a “naturally occurring nucleobase” is adenine (A), thymine (T), cytosine (C), uracil (U), and guanine (G). As used herein, a modified nucleobase is a group of atoms capable of pairing with at least one naturally occurring nucleobase. A universal base is a nucleobase that can pair with any one of the five unmodified nucleobases. As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety.

As used herein, “oligomeric compound” means a compound consisting of an oligonucleotide and optionally one or more additional features, such as a conjugate group or terminal group.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds, i.e., salts that retain the desired biological activity of the parent compound and do not impart undesired toxicological effects thereto.

As used herein “pharmaceutical composition” means a mixture of substances suitable for administering to a subject. For example, a pharmaceutical composition may comprise an antisense compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a differentform within the body or cells thereof. Typically conversion of a prodrug within the body is facilitated by the action of an enzymes (e.g., endogenous or viral enzyme) or chemicals present in cells or tissues and/or by physiologic conditions.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense oligonucleotides that act through RNase H.

As used herein, the term “single-stranded” in reference to an antisense compound and/or antisense oligonucleotide means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex.

As used herein, “sugar moiety” means an unmodified sugar moiety or a modified sugar moiety. As used herein, “unmodified sugar moiety” means a 2′-OH(H) ribosyl moiety, as found in RNA (an “unmodified RNA sugar moiety”), or a 2′-H(H) moiety, as found in DNA (an “unmodified DNA sugar moiety”). As used herein, “modified sugar moiety” or “modified sugar” means a modified furanosyl sugar moiety or a sugar surrogate. As used herein, modified furanosyl sugar moiety means a furanosyl sugar comprising a non-hydrogen substituent in place of at least one hydrogen of an unmodified sugar moiety. In certain embodiments, a modified furanosyl sugar moiety is a 2′-substituted sugar moiety. Such modified furanosyl sugar moieties include bicyclic sugars and non-bicyclic sugars. As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” mean a nucleic acid that an antisense compound is designed to affect.

As used herein, “target region” means a portion of a target nucleic acid to which an antisense compound is designed to hybridize.

As used herein, “terminal group” means a chemical group or group of atoms that is covalently linked to a terminus of an oligonucleotide.

As used here, “terminal wing nucleoside” means a nucleoside that is located at the terminus of a wing segment of a gapmer. Any wing segment that comprises or consists of at least two nucleosides has two termini: one that immediately adjacent to the gap segment; and one that is at the end opposite the gap segment. Thus, any wing segment that comprises or consists of at least two nucleosides has two terminal nucleosides, one at each terminus.

### CERTAIN EMBODIMENTS

The present disclosure includes but is not limited to the following embodiments.

I. Certain Oligonucleotides

In certain embodiments, the invention provides compounds, e.g., antisense compounds and oligomeric compounds, that comprise or consist of oligonucleotides that consist of linked nucleosides. Oligonucleotides, such as antisense oligonucleotides, may be unmodified oligonucleotides (RNA or DNA) or may be modified oligonucleotides. Modified oligonucleotides comprise at least one modification relative to unmodified RNA or DNA (i.e., comprise at least one modified nucleoside (comprising a modified sugar moiety and/or a modified nucleobase) and/or at least one modified internucleoside linkage).

A. Certain Modified Nucleosides

Modified nucleosides comprise a modified sugar moiety or a modified nucleobase or both a modified sugar moiety and a modified nucleobase.

1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including but not limited to substituents at the 2′, 4′, and/or 5′ positions. In certain embodiments, the furanosyl sugar moiety is a ribosyl sugar moiety. In certain embodiments one or more acyclic substituent of non-bicyclic modified sugar moieties is branched. Examples of 2′-substituent groups suitable for non-bicyclic modified sugar moieties include but are not limited to: 2′-F, 2′-OCH₃ (“OMe” or “O-methyl”), and 2′-O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In certain embodiments, a 2′-substituted nucleoside or 2′-non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside or 2′-non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted nucleoside or 2′-non-bicyclic modified nucleoside comprises a sugar moiety comprising a non-bridging 2′-substituent group selected from: F, OCH₃, and OCH₂CH₂OCH₃.

Nucleosides comprising modified sugar moieties, such as non-bicyclic modified sugar moieties, may be referred to by the position(s) of the substitution(s) on the sugar moiety of the nucleoside. For example, nucleosides comprising 2′-substituted or 2-modified sugar moieties are referred to as 2′-substituted nucleosides or 2-modified nucleosides.

Certain modified sugar moieties comprise a bridging sugar substituent that forms a second ring resulting in a bicyclic sugar moiety. In certain such embodiments, the bicyclic sugar moiety comprises a bridge between the 4′ and the 2′ furanose ring atoms. In certain such embodiments, the furanose ring is a ribose ring. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 20017, 129, 8362-8379; Elayadi et al.; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

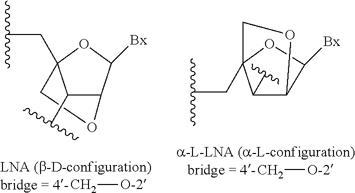

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

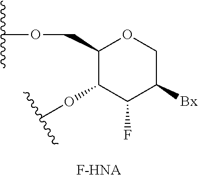

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3¹-fluoro tetrahydropyran), and nucleosides comprising additional modified THP compounds having the formula:

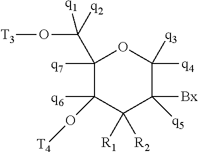

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;

q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

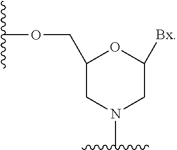

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

2. Certain Modified Nucleobases

In certain embodiments, oligonucleotides, e.g., antisense oligonucleotides, comprise one or more nucleoside comprising an unmodified nucleobase. In certain embodiments, modified oligonucleotides comprise one or more nucleoside comprising a modified nucleobase.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

B. Certain Modified Internucleoside Linkages

In certain embodiments, nucleosides of oligonucleotides, including antisense oligonucleotides, may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Representative chiral internucleoside linkages include but are not limited to alkylphosphonates and phosphorothioates. Methods of preparation of phosphorous-containing and non-phosphorous-containing internucleoside linkages are well known to those skilled in the art.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

C. Certain Motifs

In certain embodiments, modified oligonucleotides, including modified antisense oligonucleotides, comprise one or more modified nucleoside comprising a modified sugar and/or a modified nucleobase. In certain embodiments, modified oligonucleotides, including modified antisense oligonucleotides, comprise one or more modified internucleoside linkage. In such embodiments, the modified, unmodified, and differently modified sugar moieties, nucleobases, and/or internucleoside linkages of a modified oligonucleotide, such as an antisense oligonucleotide, define a pattern or motif. In certain such embodiments, the patterns or motifs of sugar moieties, nucleobases, and internucleoside linkages are each independent of one another. Thus, a modified oligonucleotide, including an antisense oligonucleotide, may be described by its sugar motif, nucleobase motif and/or internucleoside linkage motif (as used herein, nucleobase motif describes the modifications to the nucleobases independent of the nucleobase sequence).

1. Certain Sugar Motifs

In certain embodiments, oligonucleotides, including antisense oligonucleotides, comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides, such as antisense oligonucleotides, comprise or consist of a region having a gapmer motif, which comprises two external regions or “wings” and a central or internal region or “gap.” The three regions of a gapmer motif (the 5′-wing, the gap, and the 3′-wing) form a contiguous sequence of nucleosides wherein at least the sugar moieties of the terminal wing nucleosides of each of the wings differ from at least some of the sugar moieties of the nucleosides of the gap. In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, the wings of a gapmer comprise 2-5 nucleosides. In certain embodiments, the wings of a gapmer comprise 3-5 nucleosides. In certain embodiments, the nucleosides of a gapmer are all modified nucleosides.

In certain embodiments, the gap of a gapmer comprises 7-12 nucleosides. In certain embodiments, the gap of a gapmer comprises 7-10 nucleosides. In certain embodiments, the gap of a gapmer comprises 8-10 nucleosides. In certain embodiments, the gap of a gapmer comprises 10 nucleosides. In certain embodiment, each nucleoside of the gap of a gapmer is an unmodified 2′-deoxynucleoside.

The nucleosides on the gap side of each wing/gap junction are unmodified 2′-deoxyribosyl nucleosides and the nucleosides on the wing sides of each wing/gap junction are modified nucleosides. In certain such embodiments, each nucleoside of the gap is an unmodified 2′-deoxyribosyl nucleoside. In certain such embodiments, each nucleoside of each wing is a modified nucleoside.

In certain embodiments, modified oligonucleotides comprise or consist of a region having a fully modified sugar motif. In such embodiments, each nucleoside of the fully modified region of the modified oligonucleotide comprises a modified sugar moiety. In certain such embodiments, each nucleoside to the entire modified oligonucleotide comprises a modified sugar moiety. In certain embodiments, modified oligonucleotides comprise or consist of a region having a fully modified sugar motif, wherein each nucleoside within the fully modified region comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified comprises the same 2′-modification.

2. Certain Nucleobase Motifs

In certain embodiments, oligonucleotides, including antisense oligonucleotides, comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases are 5-methylcytosines.

In certain embodiments, modified oligonucleotides, such as modified antisense oligonucleotides, comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

In certain embodiments, oligonucleotides, such as antisense oligonucleotides, having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a 2′-deoxyribosyl moiety. In certain embodiments, the modified nucleobase is selected from: a 2-thiopyrimidine and a 5-propynepyrimidine.

3. Certain Internucleoside Linkage Motifs

In certain embodiments, oligonucleotides, including antisense oligonucleotides, comprise modified and/or unmodified internucleoside linkages arranged along the oligonucleotide or region thereof in a defined pattern or motif. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the sugar motif of a modified oligonucleotide is a gapmer and the internucleoside linkages within the gap are all modified. In certain such embodiments, some or all of the internucleoside linkages in the wings are unmodified phosphate linkages. In certain embodiments, the terminal internucleoside linkages are modified.

D. Certain Lengths

In certain embodiments, oligonucleotides, including antisense oligonucleotides, can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides

E. Certain Modified Oligonucleotides

In certain embodiments, the above modifications (sugar, nucleobase, internucleoside linkage) are incorporated into a modified oligonucleotide. In certain such embodiments, such modified oligonucleotides are antisense oligonucleotides. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Furthermore, in certain instances, an oligonucleotide is described by an overall length or range and by lengths or length ranges of two or more regions (e.g., regions of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists if of 15-20 linked nucleosides and has a sugar motif consisting of three regions, A, B, and C, wherein region A consists of 2-6 linked nucleosides having a specified sugar motif, region B consists of 6-10 linked nucleosides having a specified sugar motif, and region C consists of 2-6 linked nucleosides having a specified sugar motif. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of the overall length of the modified oligonucleotide (20). Herein, if a description of an oligonucleotide is silent with respect to one or more parameter, such parameter is not limited. Thus, a modified oligonucleotide described only as having a gapmer sugar motif without further description may have any length, internucleoside linkage motif, and nucleobase motif. Unless otherwise indicated, all modifications are independent of nucleobase sequence.

F. Nucleobase Sequence

In certain embodiments, oligonucleotides, such as antisense oligonucleotides, are further described by their nucleobase sequence. In certain embodiments, oligonucleotides have a nucleobase sequence that is complementary to a second oligonucleotide or a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

II. Certain Oligomeric Compounds

In certain embodiments, the invention provides oligomeric compounds, which consist of an oligonucleotide (e.g., a modified, unmodified, and/or antisense oligonucleotide) and optionally one or more conjugate groups and/or terminal groups. In certain embodiments, an oligomeric compound is also an antisense compound. In certain embodiments, an oligomeric compound is a component of an antisense compound. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

1. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

2. Conjugate Linkers

Conjugate moieties are attached to oligonucleotides through conjugate linkers. In certain compounds comprising oligonucleotides, such as oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosidesln certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides.

In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

In certain embodiments, compounds of the invention are single-stranded. In certain embodiments, oligomeric compounds are paired with a second oligonucleotide or oligomeric compound to form a duplex, which is double-stranded.

III. Certain Antisense Compounds

In certain embodiments, the present invention provides antisense compounds, which comprise or consist of an oligomeric compound comprising an antisense oliognucleotide. In certain embodiments, antisense compounds are single-stranded. Such single-stranded antisense compounds typically comprise or consist of an oligomeric compound that comprises or consists of an antisense oligonucleotide and optionally a conjugate group. In certain embodiments, antisense compounds are double-stranded. Such double-stranded antisense compounds comprise a first oligomeric compound having a region complementary to a target nucleic acid and a second oligomeric compound having a region complementary to the first oligomeric compound. The first oligomeric compound of such double stranded antisense compounds typically comprises or consists of an antisense oligonucleotide and optionally a conjugate group. The oligonucleotide of the second oligomeric compound of such double-stranded antisense compound may be modified or unmodified. Either or both oligomeric compounds of a double-stranded antisense compound may comprise a conjugate group. The oligomeric compounds of double-stranded antisense compounds may include non-complementary overhanging nucleosides.

In certain embodiments, oligomeric compounds of antisense compounds are capable of hybridizing to a target nucleic acid, resulting in at least one antisense activity. In certain embodiments, antisense compounds selectively affect one or more target nucleic acid. Such selective antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, the invention provides antisense compounds that are sufficiently “DNA-like” to elicit RNase H activity. Further, in certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain such embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain such embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, and/or a phenotypic change in a cell or animal. In certain such embodiments, the target nucleic acid is a target mRNA.

IV. Certain Target Nucleic Acids

In certain embodiments, antisense compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is a mRNA. In certain such embodiments, the target region is entirely within an exon. In certain embodiments, the target region spans an exon/exon junction. In certain embodiments, antisense compounds are at least partially complementary to more than one target nucleic acid.

A. Complementarity/Mismatches to the Target Nucleic Acid

In certain embodiments, antisense compounds comprise antisense oligonucleotides that are complementary to the target nucleic acid over the entire length of the oligonucleotide. In certain embodiments, such oligonucleotides are 99% complementary to the target nucleic acid. In certain embodiments, such oligonucleotides are 95% complementary to the target nucleic acid. In certain embodiments, such oligonucleotides are 90% complementary to the target nucleic acid. In certain embodiments, such oligonucleotides are 85% complementary to the target nucleic acid. In certain embodiments, such oligonucleotides are 80% complementary to the target nucleic acid. In certain embodiments, antisense oligonucleotides are at least 80% complementary to the target nucleic acid over the entire length of the oligonucleotide and comprise a region that is 100% or fully complementary to a target nucleic acid. In certain such embodiments, the region of full complementarity is from 6 to 20 nucleobases in length. In certain such embodiments, the region of full complementarity is from 10 to 18 nucleobases in length. In certain such embodiments, the region of full complementarity is from 18 to 20 nucleobases in length.

In certain embodiments, oligonucleotides comprise one or more mismatched nucleobases relative to the target nucleic acid. In certain such embodiments, antisense activity against the target is reduced by such mismatch, but activity against a non-target is reduced by a greater amount. Thus, in certain such embodiments selectivity of the antisense compound is improved. In certain embodiments, the mismatch is specifically positioned within an oligonucleotide having a gapmer motif. In certain such embodiments, the mismatch is at position 1, 2, 3, 4, 5, 6, 7, or 8 from the 5′-end of the gap region. In certain such embodiments, the mismatch is at position 9, 8, 7, 6, 5, 4, 3, 2, 1 from the 3′-end of the gap region. In certain such embodiments, the mismatch is at position 1, 2, 3, or 4 from the 5′-end of the wing region. In certain such embodiments, the mismatch is at position 4, 3, 2, or 1 from the 3′-end of the wing region.

V. Certain Pharmaceutical Compositions

In certain embodiments, the present invention provides pharmaceutical compositions comprising one or more antisense compound or a salt thereof. In certain such embodiments, the pharmaceutical composition comprises a suitable pharmaceutically acceptable diluent or carrier. In certain embodiments, a pharmaceutical composition comprises a sterile saline solution and one or more antisense compound. In certain embodiments, such pharmaceutical composition consists of a sterile saline solution and one or more antisense compound. In certain embodiments, the sterile saline is pharmaceutical grade saline. In certain embodiments, a pharmaceutical composition comprises one or more antisense compound and sterile water. In certain embodiments, a pharmaceutical composition consists of one antisense compound and sterile water. In certain embodiments, the sterile water is pharmaceutical grade water. In certain embodiments, a pharmaceutical composition comprises one or more antisense compound and phosphate-buffered saline (PBS). In certain embodiments, a pharmaceutical composition consists of one or more antisense compound and sterile PBS. In certain embodiments, the sterile PBS is pharmaceutical grade PBS.

In certain embodiments, pharmaceutical compositions comprise one or more or antisense compound and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, antisense compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an antisense compound encompass any pharmaceutically acceptable salts of the antisense compound, esters of the antisense compound, or salts of such esters. In certain embodiments, pharmaceutical compositions comprising antisense compounds comprising one or more antisense oligonucleotide, upon administration to an animal, including a human, are capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an antisense compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain.

VI. Certain Combinations and Combination Therapies

In certain embodiments, methods provided herein comprise administering or contacting a cell with an antisense compound (first agent) and an EGFR modulator (second agent). In certain such embodiments, the second agent increases the activity of the first agent in a cell or individual relative to the activity of the first agent in a cell or individual in the absence of the second agent. In certain embodiments, co-administration of the first and second agents permits use of lower dosages than would be required to achieve a therapeutic or prophylactic effect if the agents were administered as independent therapies.

In certain embodiments, an antisense compound comprising or consisting of an antisense oligonucleotide is co-administered with one or more EGFR modulators. In certain such embodiments, the antisense compound and one or more EGFR modulators are administered at different times. In certain embodiments, the antisense compound and one or more EGFR modulators are prepared together in a single formulation. In certain embodiments, the antisense compound and one or more EGFR modulators are prepared separately. In certain embodiments, the one or more EGFR modulators is a modified oligonucleotide complementary to the 5′-UTR of an EGFR mRNA, epidermal growth factor (EGF), transforming growth factor (TGF), TGF alpha, betacellulin, heparin-binding EGF, amphiregulin, epigen, epiregulin, or other EGFR modulator.

In certain embodiments, an antisense compound comprising or consisting of an antisense oligonucleotide and one or more EGFR modulators are used in combination treatment by administering the antisense compound and EGFR modulator simultaneously, separately, or sequentially. In certain embodiments, they are formulated as a fixed dose combination product. In other embodiments, they are provided to the patient as separate units which can then either be taken simultaneously or serially (sequentially).

### Nonlimiting Disclosure and Incorporation by Reference

While certain compounds, compositions and methods described herein have been described with specificity in accordance with certain embodiments, the following examples serve only to illustrate the compounds described herein and are not intended to limit the same. Each of the references, GenBank accession numbers, and other publications recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “ATmCGAUCG,” wherein ^{m}C indicates a cytosine base comprising a methyl group at the 5-position.

Certain compounds described herein (e.g., antisense oligonucleotides) have one or more asymmetric center and thus give rise to enantiomers, diastereomers, and other stereoisomeric configurations that may be defined, in terms of absolute stereochemistry, as (R) or (S), as a or 13 such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their racemic and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### Example 1: Protein Binding Analyses with Modified Oligonucleotides

Modified oligonucleotides in the tables below were synthesized via standard methods well known in the art. The modified oligonucleotides in Table 1 comprise a 5′- or 3′-terminal biotin tag or a 5′-terminal dye for use in the studies described below. The modified oligonucleotides in Tables 2-4 are gapmers, each with a gap containing ten 2′-deoxynucleosides, and each internucleoside linkage is a phosphorothioate internucleoside linkage. The wings of the gapmers in Table 2 each contain five 2′-MOE modified nucleosides. The wings of the gapmers in Table 3 each contain three cEt modified bicyclic nucleosides. The wings of the gapmers in Table 4 each contain five 2′-F modified nucleosides. The sequences of the modified oligonucleotides are shown in the tables below.

TABLE 1
Modified oligonucleotides

| Compound | | SEQ |
|---|---|---|
| No. | Sequence | ID No |
| 367070 | AₑₛGₑₛ^{m}CₑₛGₑₛ^{m}CₑₛA_{ds}G_{ds}A_{ds}^{m}C_{ds}A_{ds}A_{ds}A_{ds}^{m}C_{ds}^{m}C_{ds}^{m}C_{ds}AₑₛTₑₛ^{m}CₑₛAₑₛ^{m}Cₑ- | 11 |
| | TEG-Biotin | |
| 451104 | Biotin-TEG- | 14 |
| | ^{m}CₑₛTₑₛGₑₛ^{m}CₑₛTₑₛA_{ds}G_{ds}^{m}C_{ds}^{m}C_{ds}T_{ds}^{m}C_{ds}^{io}U_{ds}G_{ds}G_{ds}A_{ds}TₑₛTGₑₛTₑₛGₑₛAₑ | |
| 766636 | AF594-^{m}CₖₛTₖₛGₖₛ^{m}CₖₛTₖₛA_{ds}G_{ds}^{m}C_{ds}^{m}C_{ds}T_{ds}^{m}C_{ds}T_{ds}G_{ds}G_{ds}A_{ds}TₖₛTₖₛTₖₛGₖₛAₖ | 10 |
| 936533 | AF594-GₖₛGₖₛ^{m}C_{ds}T_{ds}A_{ds}^{m}C_{ds}T_{ds}A_{ds}^{m}C_{ds}G_{ds}^{m}C_{ds}^{m}C_{ds}G_{ds}Tₖₛ^{m}CₖₛAₖ | 15 |
| 1055615 | AF594-Gₖₛ^{m}CₖₛAₖₛT_{ds}G_{ds}T_{ds}T_{ds}^{m}C_{ds}T_{ds}^{m}Cd_{ds}A_{ds}^{m}C_{ds}A_{ds}TₖₛTₖₛAₖ | 16 |
| 1024788 | AF594-GₖₛGₖₛTₖₛ^{m}C_{ds}G_{ds}A_{ds}^{m}C_{ds}^{m}C_{ds}G_{ds}A_{ds}A_{ds}G_{ds}T_{ds}^{m}CₖₛAₖₛTₖ | 17 |
| 1024789 | AF594-^{m}Cₑₛ^{m}CₑₛTₑₛTₑₛ^{m}Cₑₛ^{m}C_{ds}^{m}C_{ds}T_{ds}G_{ds}A_{ds}A_{ds}G_{ds}G_{ds}T_{ds}T_{ds}^{m}Cₑₛ^{m}CₑₛTₑₛ^{m}Cₑₛ^{m}Cₑ | 18 |

A subscript “d” indicates an unmodified, 2′-deoxy sugar moiety. A subscript “e”
indicates a 2′-methoxyethyl modification. A subscript “k” indicates a cEt
modification. A subscript “s” indicates a phosphorothioate internucleoside
linkage. A subscript “f” indicates a 2′-F modification. A superscript “io”
before a “U” indicates 5-iodo Uracil. A superscript “m” before “C” indicates
5-methyl Cytosine. “AF594” represents Alexa Fluor 594. “TEG” represents a
tetraethylene glycol linker.

TABLE 2
5-10-5 2′-MOE modified oligonucleotides

| Compound | | SEQ |
|---|---|---|
| No. | Sequence | ID No |
| 25690 | AGGAAGGAAGCTGGCGATCT | 22 |
| 116847 | CTGCTAGCCTCTGGATTTGA | 10 |
| 395254 | GGCATATGCAGATAATGTTC | 12 |
| 395257 | CTAACATGCAATACTGCAGA | 23 |
| 462026 | CAGCAGGCAACTGTCGCTGA | 24 |
| 286529 | TTTTGGCAAAGTAATCGTCC | 25 |
| 110128 | ACACGGTATTGCCCTTGAAA | 26 |
| 324568 | TAAGTACTACTGGGCCATGG | 27 |
| 404071 | TGGTAATCCACTTTCAGAGG | 28 |
| 476366 | ACCCAATTCAGAAGGAAGGA | 29 |

TABLE 3
3-10-3 cEt modified oligonucleotides

| Compound | | SEQ |
|---|---|---|
| No. | Sequence | ID No |
| 998996 | TTCTGGGCAGCCCCAC | 30 |
| 998997 | GGCCATCACGCCACAG | 31 |
| 998998 | CTTTCCAGAGGGGCCA | 32 |
| 998999 | TCCACAGTCTTCTGGG | 33 |
| 999000 | TGGCAGTGATGGCATG | 34 |
| 999001 | GACTGTGGTCATGAGC | 35 |
| 999002 | CCTTCCACAATGCCAA | 36 |
| 999003 | AGTTGTCATGGATGAC | 37 |
| 999005 | AAGCAGTTGGTGGTGC | 38 |
| 999006 | AGGATGCATTGCTGAC | 39 |

TABLE 4
5-10-5 2′-F modified oligonucleotides

| Compound | | SEQ |
|---|---|---|
| No. | Sequence | ID No |
| 804856 | CCTTCCCTGAAGGTTCCTCC | 18 |
| 1147291 | TCGTCTGTGCATCTCTCCTG | 40 |
| 1147314 | GAGTCAGTATCCCAGTGTCT | 41 |
| 1147334 | AATCTCCTTGCTGTATTTGT | 42 |
| 1147367 | CTGATGATCTGCAGGTTTTC | 43 |
| 1147369 | AACGAGGTACTGTGTAAGTC | 44 |
| 1147360 | AUAAUCTTCCAGGGCCACAA | 45 |
| 1147366 | CUGUUGGGATATTTTAGCCU | 46 |
| 1147370 | ATATTGCATCAGATCTCAAT | 47 |

### Affinity Selection Protocol

An affinity selection method was used to identify cellular proteins that associate with modified antisense oligonucleotides comprising phosphorothioate internucleoside linkages (PS-ASOs). The PS-ASO used to capture the proteins was compound 451104 or compound 367070, which are biotinylated gapmers (see Table 1). The 5′-end of 451104 and 3′-end of 367070 are biotinylated via a tetraethyleneglycol linker. The modified oligonucleotides used to elute the proteins bound to the capture oligonucleotides were 116847, 395254, and 25690, 5-10-5 MOE gapmers; 404130, a 5-10-5 2′-fluoro gapmer; and 582801, a 5-10-5 cEt gapmer.

Agarose neutravidin beads (ThermoFisher) were incubated with compound 451104 or with biotin alone at 4° C. for 1 hr in buffer A (50 mM Tris pH 7.5, 100 mM KCl, 5 mM EDTA, 0.1% NP-40) and blocked for 30 minutes with block buffer (10 mg/ml BSA and 0.2 mg/ml tRNA in buffer A). After washing 3 times with block buffer, the PS-ASO-coated beads were incubated at 4° C. for 3 hours with 1 mg A431 cell extracts prepared in RIPA buffer [50 mM Tris-HCl pH 7.4, 150 mM NaCl, 0.5% sodium deoxycholate, 0.5 mM EDTA, protease inhibitor cocktail (Sigma)], or with 0.8 or 1.6 μg purified recombinant EGFR (PV3872, ThermoFisher Scientific). Beads were thoroughly washed with wash buffer (50 mM Tris-HCl pH 7.4, 300 mM KCl, 0.5 mM EDTA, 0.1% NP-40, 0.05% SDS). Bound proteins were eluted by incubation with 50 μL of 50 μM of a modified oligonucleotide listed in Table 1, run on SDS-PAGE, and visualized by silver staining or western blot.

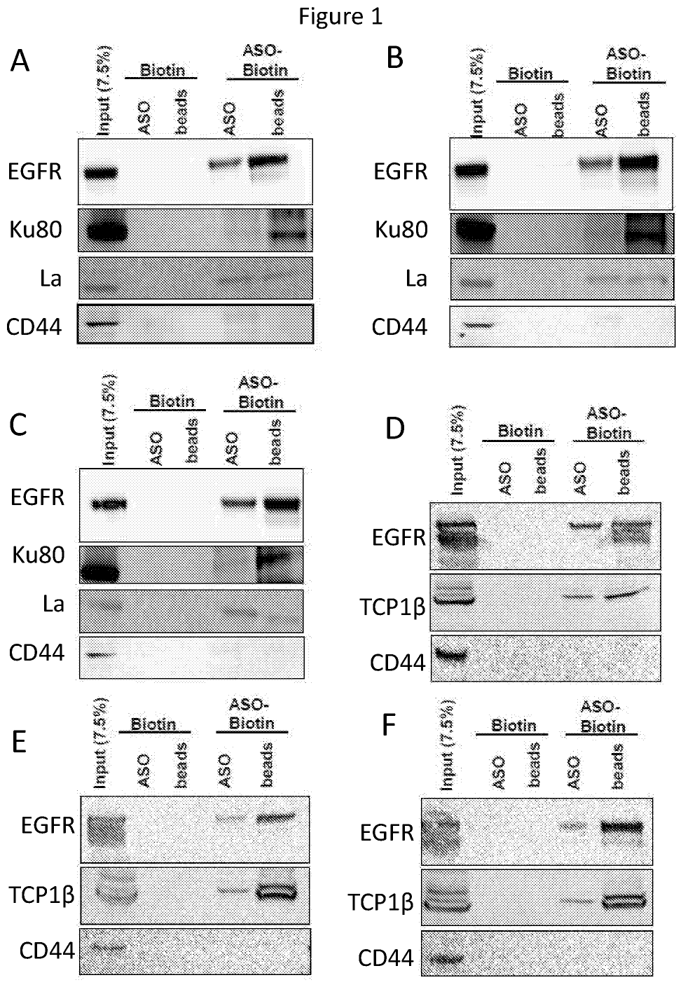

For western blots, gels were electroblotted onto PVDF membranes using the iBLOT transfer system (ThermoFisher). The membranes were blocked with 5% nonfat dry milk in PBS for 30 minutes at 4° C. Membranes were incubated with primary antibodies (EGFR: ab52894, Abcam; Ku80: 2180, Cell Signaling Technology; La: 5034, Cell Signaling Technology; CD44: ab51037, Abcam; TCP1β: sc-373769, Santa Cruz Biotech) at room temperature for 1 hour or at 4° C. overnight, and then washed three times with PBS. Membranes were then incubated with HRP-coated secondary antibodies (170-6515, Bio-Rad, 1:2,000) at room temperature for 1 hr and developed with ECL (Abcam). The results are shown in FIGS. 1-3.

FIG. 1A-C shows representative western blots for EGFR, Ku80, La, and CD44. Ku80 and La have been previously shown to associate with PS-ASO in similar assays (See Liang et al. Nucleic Acids Res. 43, 2927-2945 (2015).) Lane 1 is the cell lysate input. Beads incubated with free biotin followed by cell lysate were prepared for lanes 2 and 3. Beads incubated with PS-ASO 451104 followed by cell lysate were prepared for lanes 4 and 5. Lanes 2 and 4 show protein eluted by ASO 116847 (FIG. 1A), ASO 582801 (FIG. 1B), or ASO 404130 (FIG. 1C) from the corresponding beads, and lanes 3 and 5 show protein remaining bound to the corresponding beads.

FIG. 1D-F shows representative western blots for EGFR, TCP1β and CD44. Lane 1 is the cell lysate input fraction. Beads incubated with free biotin followed by cell lysate were prepared for lanes 2 and 3. Beads incubated with PS-ASO 451104 followed by cell lysate were prepared for lanes 4 and 5. Lanes 2 and 4 show protein eluted by ASO 116847 (FIG. 1D), ASO 395254 (FIG. 1E), or ASO 25690 (FIG. 1F) from the corresponding beads, and lanes 3 and 5 show protein remaining bound to the corresponding beads.

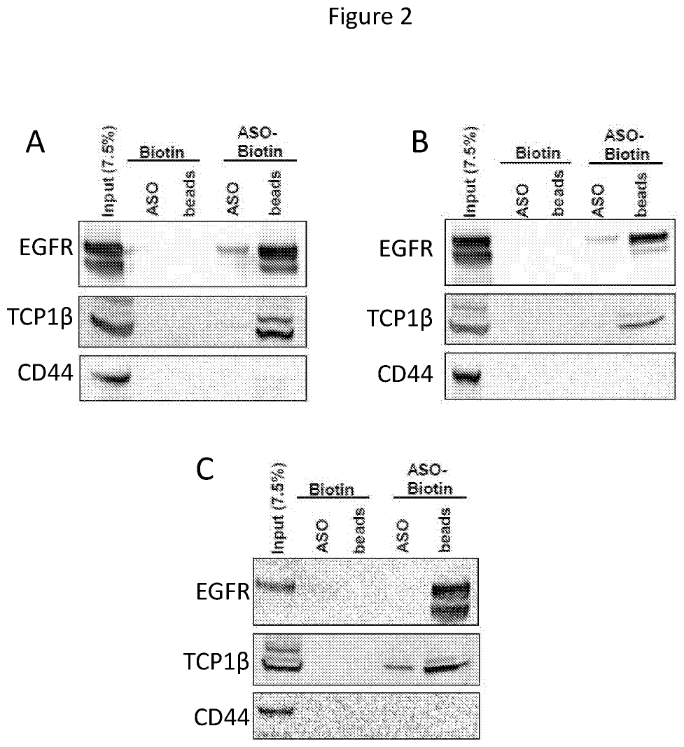

FIG. 2A-C shows representative western blots for EGFR, TCP1β and CD44. Lane 1 is the cell lysate input fraction. Beads incubated with free biotin followed by cell lysate were prepared for lanes 2 and 3. Beads incubated with PS-ASO 367070 followed by cell lysate were prepared for lanes 4 and 5. Lanes 2 and 4 show protein eluted by ASO 116847 (FIG. 2A), ASO 582801 (FIG. 2B), or ASO 404130 (FIG. 2C) from the corresponding beads, and lanes 3 and 5 show protein remaining bound to the corresponding beads.

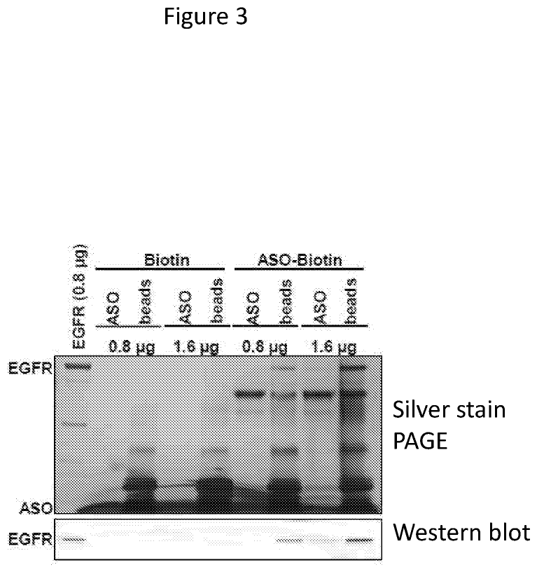

The upper panel of FIG. 3 shows a representative silver stained SDS-PAGE gel. Lane 1 is purified EGFR. Beads incubated with free biotin followed by 0.8 or 1.6 μg purified recombinant EGFR were prepared for lanes 2-5. Beads incubated with PS-ASO 451104 followed by 0.8 or 1.6 μg purified recombinant EGFR were prepared for lanes 6-9. Lanes 2, 4, 6, and 8 show protein eluted by ASO 116847 from the corresponding beads. Lanes 3, 5, 7, and 9 show protein remaining bound to the corresponding beads. The lower panel of FIG. 3 shows a representative western blot for EGFR of the same samples shown in theupper panel.

These results show that EGFR binds to PS-ASOs with a variety of sequences and modified sugar moieties.

### BRET Protocol

NanoBRET (bioluminescence resonance energy transfer) binding assays were performed as described in Vickers and Crooke. PLOS One, 11(8), (2016). An EGFR NLuc construct was prepared by first amplifying human EGFR from the full length cDNA clone (Origene RC217223) with forward PCR primer 5′-GCTAGCAGCCACCATGCGACCCTCCGGGACG-3′ (SEQ ID NO: 1) and reverse PCR primer 5′-GCGCCACATCGTTCGGAAGGACTCGAG (SEQ ID NO: 2). The amplified product was ligated into the NheI and XhoI sites of the NanoLuc expression vector pFC32K Nluc CMV-Neo (Promega). Protein was expressed in HEK293 cells and isolated using Protein G magnetic beads. For competitive BRET, the Alexafluor594-labeled modified oligonucleotide 766636 was diluted into water in opaque white 96-well plates at 10 nM and competed with 0.1-10,000 nM of unlabeled modified oligonucleotide. 50 4/well of 2× binding buffer containing 106 RLU (relative luminescence units) beads/well was added and plates were shaken for 10 minutes at room temperature. Nanoluciferase activity and BRET were measured in a Glowmax Discover plate reader and EC50 values, shown in the tables below, were calculated using GraphPad Prism. For direct BRET, Alexafluor594 modified nucleotides were diluted at 0.1-10,000 nM and experiments were performed as described above. The results show that PS-ASOs with various modified sugars bound to purified EGFR and that the cEt containing PS-ASOs bound most tightly.

TABLE 5
Competitive BRET

| Compound No. | Wing chemistry | EC₅₀ (nM) |
|---|---|---|
| 404130 | 2′F | 158 |
| 116847 | MOE | 181 |
| 582801 | cEt | 42.7 |
| 1069848 (polyA) | cEt | >10,000 |
| 1069849 (polyC) | cEt | 2025 |
| 1069850 (polyT) | cEt | 220 |

TABLE 6
Competitive BRET

| Compound No. | Wing chemistry | EC₅₀ (nM) |
|---|---|---|
| 25690 | 2′-MOE | 1454 |
| 395254 | 2′-MOE | 1237 |
| 116847 | 2′-MOE | 1656 |
| 395257 | 2′-MOE | 2626 |
| 462026 | 2′-MOE | 670.3 |
| 286529 | 2′-MOE | 1845 |
| 110128 | 2′-MOE | 2277 |
| 324568 | 2′-MOE | 648.1 |
| 404071 | 2′-MOE | 3137 |
| 476366 | 2′-MOE | 2237 |
| 998996 | cEt | 1434 |
| 998997 | cEt | 2796 |
| 998998 | cEt | 7.25 |
| 998999 | cEt | 213.6 |
| 999000 | cEt | 1404 |
| 999001 | cEt | 2336 |
| 999002 | cEt | 632.7 |
| 999003 | cEt | 2301 |
| 999005 | cEt | 39.11 |
| 999006 | cEt | 1708 |
| 998996 | cEt | 1434 |
| 998997 | cEt | 2796 |
| 804856 | 2′-F | 2750 |
| 1147369 | 2′-F | 2626 |
| 1147367 | 2′-F | 113.8 |
| 1167370 | 2′-F | 4652 |
| 1167366 | 2′-F | 87.25 |
| 1167360 | 2′-F | 108 |
| 1147334 | 2′-F | 4371 |
| 1147314 | 2′-F | 217.8 |
| 1147291 | 2′-F | 164.7 |

TABLE 7
Direct BRET

| Compound No. | Wing chemistry | EC₅₀ (M) |
|---|---|---|
| 936533 | cEt | 5.511e−007 |
| 1055615 | cEt | 7.465e−007 |
| 1024788 | cEt | 5.251e−007 |
| 1024789 | MOE | 2.02e−007 |

### Example 2: Immunofluorescent Microscopy

Immunofluorescent staining was used to visualize clathrin, EGFR, and PS-ASOs in A431 cells. Compound no. 446654 has the sequence and structure Cy3-^{m}CₑₛTₑₛGₑₛ^{m}CₑₛTₑₛA_{ds}G_{ds}^{m}C_{ds}T_{ds}^{m}C_{ds}T_{ds}G_{ds}G_{ds}A_{ds}TₑₛTₑₛTₑₛGₑₛAₑ (SEQ ID NO: 10). Cells were incubated with FITC labeled epidermal growth factor (EGF) or unlabeled EGF and compound no. 446654 for 30 minutes, then fixed with 4% paraformaldehyde for 20 minutes at room temperature and permeabilized with 0.05% saponin (Sigma) in PBS for 5 minutes. Cells were treated with blocking buffer (1 mg/mL BSA in PBS) for 30 minutes and then incubated with primary antibodies at room temperature for 2-4 hours or at 4° C. overnight. Primary antibodies used were ab30 (Abcam) for EGFR, ab21679 (Abcam) for clathrin, and antibody 610456 (BD Bioscience) for early endosome antigen 1 (EEA1). After three washes with PBS, cells were fluorescently labeled with secondary antibodies at room temperature for 1-2 hours. Secondary antibodies used were anti-mouse conjugated to AF488 (ab150077, Abcam) or AF647 (ab150079, Abcam), and anti-rabbit conjugated to AF488 (ab150113, Abcam) or AF647 (ab150115, Abcam). Nuclei were labeled with Hoechst 33342. Cells were then visualized with a confocal microscope (Olympus FV-1000), and single slices and Z-stack images were obtained. Co-localization between PS-ASOs and different organelles was analyzed using FV10-ASW 3.0 viewer software.

In cells incubated with unlabeled EGF, compound no. 446654, and antibodies to detect EGFR and clathrin, co-localization of EGFR and compound no. 446654 was observed both at the cell surface and within the cytoplasm. Co-localization of EGFR and clathrin was observed at the cell surface. Co-localization of compound no. 446654 and clathrin was observed at the cell surface.

In cells incubated with FITC-EGF, compound no. 446654, and antibodies to detect EGFR and clathrin, co-localization of EGF and compound no. 446654 was observed within the cytoplasm and at the cell surface. Co-localization of FITC-EGF and clathrin was observed at the cell surface. Co-localization of compound no. 446654 and clathrin was observed at the cell surface.

In cells incubated with unlabeled EGF, compound no. 446654, and antibodies to detect EGFR and EEA1, co-localization of EGFR and compound no. 446654 was observed at the cell surface, in the cytoplasm, and within the nucleus. Co-localization of EGFR and EEA1 was observed in the cytoplasm and within the nucleus. Co-localization of compound no. 446654 and EEA1 was observed in the cytoplasm and within the nucleus.

In cells incubated with FITC-EGF, compound no. 446654, and antibodies to detect EGFR and EEA1, co-localization of EGF and compound no. 446654 was observed both at the cell surface and within the cytoplasm. Co-localization of EGFR and EEA1 was observed within the cytoplasm. Co-localization of compound no. 446654 and EEA1 was observed within the cytoplasm.

These observations were consistent across both images of single slices and Z-stack images and show that a PS-ASO was internalized as cargo together with EGF and EGFR in clathrin-containing vesicles.

### Example 3: Immunofluorescent Microscopy of Cells with Enlarged Endosomes

Cells with enlarged endosomes were created by overexpressing a constitutively active form of Rab5, Rab5(Q79L)-GFP in A431 cells (See Ceresa et al. J. Biol. Chem. 276, 9649-9654 (2001)). These cells were treated with Cy3-labeled compound no. 446654, unlabeled EGF, and/or Alexa Fluor 647-EGF for four hours prior to immunostaining for EGFR as described in Example 2. The cells were visualized in single slices and Z-stacks, as described in Example 2.

In Rab5(Q79L)-GFP cells incubated with Alexa Fluor 647-EGF in the absence of a PS-ASO, co-localization between EGF and EGFR and colocalization was observed. In Rab5(Q79L)-GFP cells incubated with Alexa Fluor 647-EGF in the presence of PS-ASO compound no. 446654, co-localization between between EGF and compound no. 446654 was observed. In Rab5(Q79L)-GFP cells incubated with unlabeled EGF in the presence of PS-ASO compound no. 446654, co-localization between EGFR and compound no. 446654 was observed. The co-localization between the PS-ASO compound no. 446654 and EGFR or EGF was not as substantial as that between EGFR and EGF in the enlarged endosomes. These observations show that cellular uptake of PS-ASOs may be mediated in part by EGFR.

### Example 4: Membrane Protein Binding Assay

TABLE 8
Modified Oligonucleotides

| Compound | | SEQ |
|---|---|---|
| No | Sequence | ID No |
| 256903 | FITC-^{m}CₑₛTₑₛGₑₛ^{m}CₑₛTₑₛA_{ds}G_{ds}^{m}C_{ds}^{m}C_{ds}T_{ds}^{m} | 10 |
| | C_{ds}T_{ds}G_{ds}G_{ds}A_{ds}TₑₛTₑₛTₑₛGₑₛAₑ | |
| PO-ASO | FITC-^{m}CₑₒTₑₒGₑₒ^{m}CₑₒTₑₒA_{do}G_{do}^{m}C_{ds}^{m}C_{do}T_{do}^{m} | 10 |
| | C_{do}T_{do}G_{do}G_{do}A_{do}TₑₒTₑₒTₑₒGₑₒAₑ | |

A subscript “d” indicates an unmodified, 2′-deoxy sugar
moiety. A subscript “e” indicates a 2′-methoxyethyl
modification. A subscript “s” indicates a phosphorothioate
linkage and a subscript “o” indicates a phosphate
internucleoside linkage. A superscript “m” indicates
5-methyl cytosine.

A membrane binding assay was performed to test the binding affinities of modified oligonucleotides to EGF and EGFR. Purified recombinant EGF (PHG0311L, ThermoFisher) or EGFR protein (PV3872, ThermoFisher) were incubated with FITC-labeled phosphorothioate oligonucleotide, compound no. 256903 or FITC-labeled phosphate oligonucleotide, PO-ASO, in binding buffer (20 mM Tris-HCl, pH 7.5, 150 mM NaCl, 1 mM DTT, 10% glycerol) for 1 hr at 37° C. Each reaction contained purified EGF at 3 nM to 3 μM or recombinant EGFR at 5 nM to 150 nM. Samples were loaded onto a hyband ECL nitrocellulose membrane (GE Healthcare) and soaked in wash buffer (20 mM Tris-HcCl, pH 7.5, 250 mM NaCl). Protein-bound ASOs were transferred to the membrane by applying a vaccum in a 96-well Bio-Rad Bio-Dot apparatus. After washing, membranes were air-dried and scanned using a phoshoimager (GE Healthcare). The signal intensities were quantified using ImageJ, and the resulting relative intensities are shown in the tables below. K_{d}s were calculated for compound no. 256903 using Prism. The results below represent an average of three replicate experiments. The PO-ASO did not appreciably interact with either EGF or EGFR, although a faint signal was observed for PO-ASO at the highest EGF concentration. In contrast, compound no. 256903 bound to both EGF and EGFR, with a higher affinity for EGFR than for EGF.

TABLE 9
Binding affinity of compound no. 256903 for EGF

| [EGF] (nM) | Relative intensity (%) |
|---|---|
| 0 | 0 |
| 2.5 | 8.8 |
| 5 | 10.2 |
| 10 | 12.0 |
| 20.9 | 12.8 |
| 41.9 | 13.7 |
| 93.8 | 15.7 |
| 187.5 | 17.0 |
| 375 | 22.3 |
| 750 | 72.3 |
| 1500 | 95.2 |
| 3000 | 97.3 |
| K_{d} | 0.57 ± 0.17 μM |

TABLE 10
Binding affinity of compound no. 256903 for EGFR

| [EGF] (nM) | Relative intensity (%) |
|---|---|
| 0 | 0 |
| 2.3 | 7.0 |
| 4.7 | 9.0 |
| 9.4 | 9.0 |
| 18.8 | 10.3 |
| 37.5 | 40.5 |
| 75 | 80.0 |
| 150 | 102.7 |
| K_{d} | 51.5 ± 9.3 nM |

### Example 5: Pull-Down Assay in the Presence of EGF

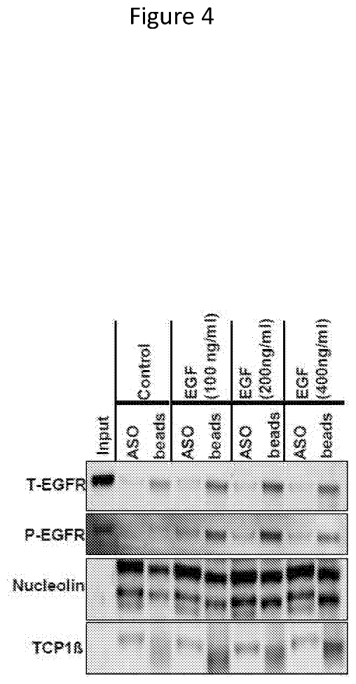

A431 cells were treated with 100 ng/mL, 200 ng/mL, or 400 ng/mL EGF and then lysed. The cell lysates were mixed with beads bound to compound no. 451104, as prepared as in Example 1. Proteins were eluted with compound no. 116847 and run on a SDS-PAGE followed by western blot, as in Example 1. The same membrane was sequentially blotted for total EGFR (T-EGFR), phosphorylated EGFR (P-EGFR, ab205827, Abcam), nucleolin (ab22758, Abcam), and TCP1β. FIG. 4 shows the four resulting blots. Lane 1 shows the cell lysate input alone, lanes 2 and 3 show ASO elution and bead bound sample from control cells not treated with EGF, and lanes 4-9 show ASO elution and bead bound samples from cells treated with varying concentrations of EGF, as shown. The results show that exogenous EGF did not compete for the binding of compound no. 451104 to EGFR.

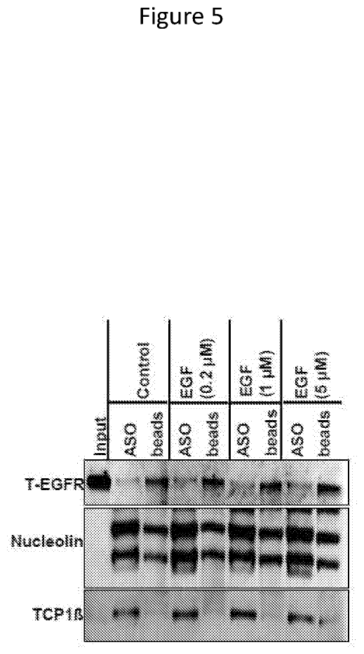

In a similar experiment in which EGF was added after cell lysis, A431 cells were lysed and mixed with beads bound to compound no. 451104. Varying concentrations of EGF were added during the elution step with compound no. 116847. The resulting western blots are shown in FIG. 5. The results show that direct addition of EGF to the cell lysates did not significantly alter the recovery of EGFR, nucleolin, or TCP1β from the beads.

### Example 6: Competitive BRET

The binding affinities for EGFR of PS-ASOs with various sugar modifications were measured with competitive BRET, as described in Example 1. 10 nM compound no. 766636 was competed with 0.1 to 3,000 nM of an unconjugated modified oligonucleotide listed in the table below in the absence of EGF or in the presence of 100 ng/mL exogenous EGF.

TABLE 11
BRET

| | EGF added | | EGF added | | EGF added | |
|---|---|---|---|---|---|---|
| | 0 | 100 ng/mL | 0 | 100 ng/mL | 0 | 100 ng/mL |
| | Compound No. | | Compound No. | | Compound No. | |
| [Compound] | 2′-F (404130) | | cEt (582801) | | 2′-MOE (116847) | |
| (nM) | Bret Ratio (% Max) | | | | | |
| 0.01 | 6.3 | 7.1 | 9.2 | 9.9 | 15.0 | 13.3 |
| 0.03 | 9.4 | 8.7 | 10.3 | 11.0 | 15.4 | 12.4 |
| 0.1 | 11.5 | 10.2 | 12.0 | 11.8 | 16.2 | 13.2 |
| 0.30 | 14.9 | 11.4 | 14.3 | 11.9 | 19.6 | 15.1 |
| 1.0 | 21.8 | 14.8 | 17.0 | 16.2 | 26.1 | 18.0 |
| 3.0 | 30.0 | 17.9 | 23.0 | 22.7 | 32.6 | 24.2 |
| 10.0 | 41.2 | 27.3 | 33.5 | 30.6 | 41.8 | 30.7 |
| 30.0 | 54.1 | 39.4 | 55.4 | 44.0 | 52.8 | 39.7 |
| 100 | 64.9 | 58.3 | 72.6 | 62.4 | 64.1 | 54.7 |
| 300 | 74.2 | 68.9 | 87.5 | 69.7 | 70.8 | 63.8 |
| 1000 | 89.2 | 83.2 | 94.1 | 85.0 | 82.9 | 77.4 |
| 3000 | 102.4 | 99.4 | 99.9 | 99.6 | 99.5 | 94.2 |

### Example 7: Effect of PS-ASOs on EGFR

TABLE 12
Modified oligonucleotides

| Compound | | SEQ |
|---|---|---|
| No | Sequence | ID No |
| 110080 | ^{m}CₑₛGₑₛTₑₛ^{m}CₑₛGₑₛT_{ds}^{m}C_{ds}G_{ds}T_{ds}^{m}C_{ds}A_{ds}T_{ds}^{m}C_{ds}^{m} | 48 |
| | C_{ds}T_{ds}^{m}CₑₛGₑₛTₑₛ^{m}Cₑₛ^{m}Cₑ | |
| 25699 | AₑₛGₑₛTₑₛ^{m}CₑₛTₑₛA_{ds}G_{ds}G_{ds}G_{ds}T_{ds}^{m}C_{ds}A_{ds}^{m}C_{ds}A_{ds} | 49 |
| | A_{ds}Tₑₛ^{m}CₑₛTₑₛGₑₛGₑ | |
| 395251 | ^{m}Cₑₛ^{m}CₑₛAₑₛGₑₛGₑₛ^{m}C_{ds}T_{ds}G_{ds}G_{ds}T_{ds}T_{ds}A_{ds}T_{ds}G_{ds} | 50 |
| | A_{ds}^{m}CₑₛTₑₛ^{m}CₑₛAₑₛGₑ | |

A subscript “d” indicates an unmodified, 2′-deoxy sugar
moiety. A subscript “e” indicates a 2′-methoxyethyl
modification. A subscript “s” indicates a phosphorothioate
linkage and a subscript “o” indicates a phosphate
internucleoside linkage. A superscript “m” indicates
5-methyl cytosine.

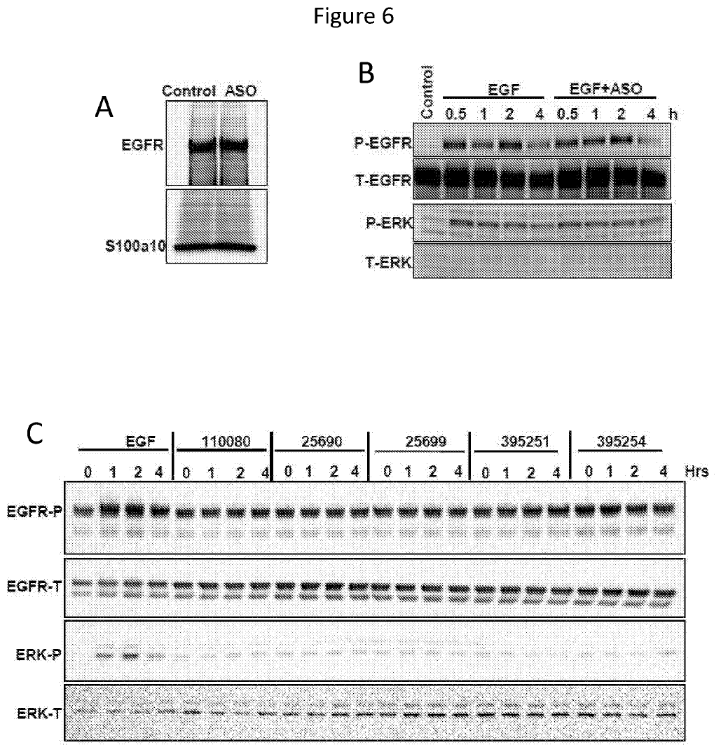

In order to test the effects of a PS-ASO on EGFR synthesis and degradation, A431 cells were incubated with either compound no. 116847 or no PS-ASO compound for 16 hours. A pulse-chase protocol was then performed in which the cells were incubated for 20 minutes in cysteine and methionine free media followed by incubation with [³⁵S]-Met and [³⁵S]-Cys in order to analyze newly synthesized protein. Cell samples were collected in RIPA buffer after 50 minutes (FIG. 6A) or at the times indicated in the tables below, and cell lysates were immunoprecipitated with EGFR antibody or s100a10 antibody (610071, BD Bioscience). The resulting, labeled immunocomplexes were resolved by SDS-PAGE and visualized by autoradiography using phosphoimager. FIG. 6A and Table 13 show the levels of nascent EGFR protein and nascent s100a10, a control protein. The results show that EGFR synthesis and degradation were unchanged in cells incubated with compound no. 116847 relative to cells that were not incubated with an ASO.

TABLE 13
Relative Abundance of EGFR (%)

| | EGFR (%) | |
|---|---|---|
| Time (hr) | Control | 116847 |
| 0 | 100 | 103 |
| 4 | 89 | 91 |
| 16 | 72 | 69 |
| 24 | 55 | 54 |

In order to test the effects of a PS-ASO on EGFR signaling, A431 cells were incubated with compound no. 116847 or no PS-ASO compound for 16 hours. All cells were then treated with EGF prior to being subjected to the pulse-chase protocol described above. Cell lysates were run on a SDS-PAGE gel and analyzed by sequential western blot for phosphorylated EGFR (P-EGFR), total EGFR (T-EGFR), phosphorylated ERK (P-ERK), and total ERK (T-ERK) using the antibodies described above for EGFR, 4370 for P-ERK (Cell Signaling Technology), and 4695 for T-ERK (Cell Signaling Technology). FIG. 6B shows the resulting western blot, which indicates that EGF-EGFR signaling was not affected by the presence of a PS-ASO. The western blot was quantified using ImageLab (Bio-Rad) and the data for p-EGFR and p-ERK are presented in Table 14.

TABLE 14
Relative Abundance of p-EGFR and p-ERK

| | p-EGFR (%) | | p-ERK (%) | |
|---|---|---|---|---|
| Time (hr) | Control | 116847 | Control | 116847 |
| 0.5 | 100 | 89 | 100 | 102 |
| 1 | 78 | 76 | 92 | 94 |
| 2 | 91 | 85 | 83 | 85 |
| 4 | 65 | 55 | 79 | 87 |

In order to test for confirmation of these results with additional PS-ASOs, A431 cells were incubated with EGF (as a positive control), compound no. 110080, 25690, 25699, 395251, or 395254 at 2 μM for 16 hours prior to carrying out the pulse-chase experiment described above. The results are presented in FIG. 6C and show that EGFR signaling was confirmed to not be impacted by any of the tested PS-ASOs.

Microscopy studies were also carried out to evaluate the EGF-induced internalization and recycling of EGFR in the presence of Cy3-labeled PS-ASO, compound no. 446654. Cells were treated with EGF alone or EGF and compound no. 446654 for 16 hours. The cells were then either immediately stained and imaged, or the EGF and, if applicable, compound no. 446654 were removed for two hours prior to staining and imaging. Staining and imaging were performed as described above. In cells treated with EGF alone, microscopy images at 16 hours showed punctuate distribution of EGFR in the cytoplasm of cells as well as staining of the plasma membrane. Two hours after removal of EGF, cells show EGFR staining primarily in the plasma membrane. In cells treated with Cy3-labeled ASO (compound no. 446654) and EGF, the ASO was distributed throughout the cytoplasm, and EGFR distribution was similar to that observed upon treatment with EGF alone. Two hours after removal of EGF and/or compound no. 446654, the Cy3 signal remained diffuse throughout the cytoplasm, while EGFR staining primarily localized to the plasma membrane. These results are consistent with the observations that PS-ASOs did not affect EGFR and that productive uptake of PS-ASOs was increased in the presence of EGFR internalization.

### Example 8: Effects of Growth Factors on PS-ASO Antisense Activity and Uptake

A431 cells were grown at 10,000 cells per well and pre-treated with a variety of growth factors to determine if the addition of exogenous growth factors affects antisense activity or uptake of modified antisense oligonucleotides in a cell. The growth factors used were EGF, insulin growth factor (IGF), transforming growth factor (TGF), vesicular endothelial growth factor (VEGF), hepatocyte growth factor (HGF), platelet growth factor (PGF) and fibroblasts growth factor (FGF). To test antisense activity, cells were treated for 4 hours with a growth factor or vehicle alone, and then treated with compound no. 25690 (complementary to Drosha), compound no. 395254 (complementary to Malat1), or vehicle alone. RNA was isolated from the cells and Drosha or Malat1 RNA levels were measured by RT-qPCR. Human primer probe set 13816 (forward sequence CAAGCTCTGTCCGTATCGATCA, designated herein as SEQ ID NO: 3; reverse sequence TGGACGATAATCGGAAAAGTAATCA, designated herein as SEQ ID NO: 4; probe sequence CTGGATCGTGAACAGTTCAACCCCGAT, designated herein as SEQ ID NO: 5) was used to measure Drosha mRNA levels and primer probe set RTS2736 (forward sequence AAAGCAAGGTCTCCCCACAAG, designated herein as SEQ ID NO: 6; reverse sequence TGAAGGGTCTGTGCTAGATCAAAA, designated herein as SEQ ID NO: 7; probe sequence TGCCACATCGCCACCCCGT, designated herein as SEQ ID NO: 8) was used to measure Malat1 RNA levels. Drosha and Malat1 RNA levels were normalized to total RNA content, as measured by RIBOGREEN®. Results are presented in the tables below as normalized RNA levels, relative to untreated control cells. The results indicate that EGF and TGF both increased antisense activity relative to the activity observed in the absence of any growth factor. Results for IGF, FGF, HGF, VEGF, and PGF showed that the half maximal inhibitory concentrations of the PS-ASOs 25690 and 395254 were unchanged in cells treated with them relative to cells treated with the PS-ASOs alone (data not shown).

Uptake of compound no. 446654 by A431 cells was measured by flow cytometry. A431 cells were treated with a growth factor for 4 hours prior to incubation with compound no. 446654 for 2 hours. Results are presented in the tables below as relative fluorescence units (RFU) and represent an average of three independent experiments. The results indicate that total uptake was not affected in a significant, dose dependent manner by any growth factor treatment tested.

TABLE 15
Antisense activity of PS-ASO complementary to Drosha

| [compound no. | Drosha mRNA (% Control) | | |
|---|---|---|---|
| 25690] (nM) | Control | EGF | TGF |
| 19.5 | 100.0 | 100.0 | 100.0 |
| 39 | 104.3 | 98.0 | 95.1 |
| 78 | 100.3 | 98.0 | 80.2 |
| 156.25 | 103.8 | 70.7 | 64.6 |
| 312.5 | 92.7 | 72.8 | 61.1 |
| 625 | 90.9 | 57.2 | 50.8 |
| 1250 | 75.7 | 52.8 | 43.4 |
| 2500 | 63.6 | 42.8 | 40.1 |
| 5000 | 47.6 | 33.0 | 31.9 |
| 10000 | 48.2 | 27.7 | 29.7 |
| 20000 | 48.8 | 25.4 | 25.5 |
| 40000 | 44.3 | 23.6 | 22.7 |
| IC₅₀ (μM) | 1.47 ± 0.05 | 0.60 ± 0.08 | 0.59 ± 0.07 |

TABLE 16
Antisense activity of PS-ASO complementary to Malat1

| [compound no. | Malat1 RNA (% Control) | | |
|---|---|---|---|
| 395254] (nM) | Control | EGF | TGF |
| 2.0 | 100.0 | 100.0 | 100.0 |
| 3.9 | 109.0 | 76.0 | 99.9 |
| 7.8 | 127.0 | 98.1 | 101.8 |
| 15.6 | 125.6 | 96.4 | 93.2 |
| 31.3 | 120.2 | 111.9 | 109.8 |
| 62.5 | 109.6 | 85.9 | 91.4 |
| 125 | 131.6 | 62.3 | 74.4 |
| 250 | 74.0 | 43.6 | 29.4 |
| 500 | 44.5 | 26.1 | 27.9 |
| 1000 | 27.0 | 15.1 | 14.2 |
| 2000 | 24.1 | 9.9 | 9.5 |
| 4000 | 17.9 | 10.3 | 11.6 |
| IC₅₀ (nM) | 36 ± 3.2 | 15 ± 3.0 | 13 ± 2.1 |

TABLE 17
Total uptake of Cy3-labeled ASO by flow cytometry

| [compound | | |
|---|---|---|
| no. | Growth | |
| 446654] | Factor | RFU |
| 0.12 μM | Control | 1475 |
| | EGF | 1772 |
| | FGF | 1420 |
| | VEGF | 1147 |
| | PGF | 1321 |
| | HGF | 1204 |
| | IGF | 1659 |
| | TGF | 1500 |
| 0.25 μM | Control | 1606 |
| | EGF | 1917 |
| | FGF | 1682 |
| | VEGF | 1423 |
| | PGF | 1275 |
| | HGF | 1481 |
| | IGF | 1435 |
| | TGF | 1871 |
| 0.5 μM | Control | 2223 |
| | EGF | 2349 |
| | FGF | 1827 |
| | VEGF | 1973 |
| | PGF | 2112 |
| | HGF | 2095 |
| | IGF | 1917 |
| | TGF | 2081 |
| 1.0 μM | Control | 1565 |
| | EGF | 2532 |
| | FGF | 2414 |
| | VEGF | 2655 |
| | PGF | 2487 |
| | HGF | 2533 |
| | IGF | 2237 |
| | TGF | 2826 |

### Example 9: Effects of Blocking EGFR Internalization on Antisense Activity

A431 cells were treated with EGF or TGF at 200 ng/mL in the presence or absence of 1 μM of the EGFR tyrosine kinase inhibitor PD174265. The cells were then treated with compound no. 25690 or compound no. 395254 as in Example 8. Total RNA was isolated and analyzed by RT-qPCR, as in Example 8. The results show that inhibition of EGFR blocked the growth factor mediated increase in antisense activities of multiple PS-ASOs.

TABLE 18
Antisense activity of PS-ASO complementary to Drosha

| [compound | | | |
|---|---|---|---|
| no. 25690] | Drosha mRNA (% Control) | | |
| (nM) | Control | EGF | EGF+PD174265 |
| 97.7 | 100.0 | 100.0 | 100.0 |
| 195 | 110.0 | 98.2 | 102.4 |
| 391 | 99.7 | 89.4 | 103.3 |
| 781 | 88.4 | 81.4 | 83.6 |
| 1563 | 83.2 | 74.7 | 68.9 |
| 3125 | 66.6 | 55.9 | 62.7 |
| 6250 | 66.2 | 48.4 | 64.7 |
| 12500 | 56.4 | 40.1 | 53.6 |
| 25000 | 45.2 | 40.1 | 51.2 |
| 50000 | 40.2 | 35.1 | 45.3 |
| 100000 | 39.2 | 32.9 | 35.2 |
| 200000 | 30.2 | 25.1 | 33.2 |
| IC₅₀ (μM) | 1.62 ± 0.09 | 0.58 ± 0.07 | 1.50 ± 0.05 |

TABLE 19
Antisense activity of PS-ASO complementary to Drosha

| [compound | | | |
|---|---|---|---|
| no. 25690] | Malat1 RNA (% Control) | | |
| (nM) | Control | EGF | EGF+PD174265 |
| 2.0 | 100.0 | 100.0 | 100.0 |
| 3.9 | 99.2 | 105.0 | 110.0 |
| 7.8 | 98.2 | 78.2 | 97.2 |
| 15.6 | 72.3 | 60.1 | 61.2 |
| 31.3 | 64.4 | 40.5 | 59.8 |
| 62.5 | 59.6 | 43.9 | 50.2 |
| 125 | 46.2 | 25.9 | 39.6 |
| 250 | 36.1 | 18.9 | 45.6 |
| 500 | 35.5 | 14.5 | 42.5 |
| 1000 | 22.3 | 15.2 | 22.3 |
| 2000 | 19.2 | 12.4 | 15.3 |
| 4000 | 17.2 | 10.2 | 13.2 |
| IC₅₀ (nM) | 43 ± 5.7 | 19 ± 3.0 | 47 ± 5.3 |

TABLE 20
Antisense activity of PS-ASO complementary to Drosha

| [compound | | | |
|---|---|---|---|
| no. 25690] | Drosha mRNA (% Control) | | |
| (nM) | Control | TGF | TGF+PD174265 |
| 97.7 | 100.0 | 100.0 | 100.0 |
| 195 | 110.0 | 95.2 | 108.0 |
| 391 | 99.3 | 85.2 | 91.2 |
| 781 | 87.2 | 67.2 | 87.2 |
| 1563 | 76.3 | 65.9 | 78.7 |
| 3125 | 67.2 | 47.2 | 64.2 |
| 6250 | 54.0 | 43.1 | 57.0 |
| 12500 | 47.6 | 29.7 | 44.9 |
| 25000 | 38.9 | 23.5 | 37.1 |
| 50000 | 28.2 | 17.2 | 35.2 |
| 100000 | 25.2 | 15.2 | 30.2 |
| 200000 | 21.2 | 15.2 | 27.2 |
| IC₅₀ (μM) | 1.39 ± 0.06 | 0.50 ± 0.04 | 1.49 ± 0.04 |

TABLE 21
Antisense activity of PS-ASO complementary to Malat1

| [compound | | | |
|---|---|---|---|
| no. 395254] | Malat1 RNA (% Control) | | |
| (nM) | Control | TGF | TGF+PD174265 |
| 2.0 | 100.0 | 100.0 | 100.0 |
| 3.9 | 110.2 | 105.2 | 108.2 |
| 7.8 | 99.3 | 105.2 | 91.2 |
| 15.6 | 77.2 | 107.2 | 77.2 |
| 31.3 | 66.3 | 55.9 | 68.7 |
| 62.5 | 57.2 | 47.2 | 54.2 |
| 125 | 44.0 | 33.1 | 47.0 |
| 250 | 37.6 | 19.7 | 34.9 |
| 500 | 28.9 | 13.5 | 27.1 |
| 1000 | 15.2 | 7.2 | 21.2 |
| 2000 | 11.2 | 5.2 | 10.2 |
| 4000 | 7.2 | 5.7 | 7.2 |
| IC₅₀ (nM) | 50 ± 7 | 21 ± 4.0 | 56 ± 8.1 |

### Example 10: Effects of Inhibiting EGFR Expression on Antisense Activity, Localization, and Uptake

EGFR levels in A431 cells were reduced using two siRNAs targeting EGFR, Assay ID 42833 and Assay ID 644 (ThermoFisher). A siRNA targeting luciferase was used for a control. Treatment of cells with the EGFR siRNA reduced EGFR protein levels more than 80%. Following siRNA treatment, cells were treated with additional compounds to test for antisense compound localization, activity, or uptake, as described below.

### Microscopy

Following siRNA treatment, cells were treated with compound no. 446654 for two hours. EEA1 was labeled as in Example 2 and LAMP1 was labeled with an antibody. EEA1 is a marker for early endosomes and LAMP1 is a marker for late endosomes. Co-localization of compound no. 446654 with EEA1 and with LAMP1 was observed. The number of 446654 loci co-localized with EEA1 or LAMP1 was counted in 20 cells, and compared to the total number of 446654 loci. These data are presented in the tables below. The difference observed with control siRNA treatment vs EGFR siRNA treatment shown in Table 22 is significant, as determined by the student T-test (p<0.05), whereas the difference shown in Table 23 was not determined to be significant by the student T-test.

TABLE 22
Localization of compound
no. 446654 in early endosomes

| | % |
|---|---|
| | ASO EE/ |
| siRNA treatment | Total ASO |
| Control luciferase | 15 |
| siRNA | |
| EGFR siRNA-1 | 26 |

TABLE 23
Localization of compound
no. 446654 in late endosomes

| | % |
|---|---|
| | ASO LE/ |
| siRNA treatment | Total ASO |
| Control luciferase | 79 |
| siRNA | |
| EGFR siRNA-1 | 66 |

Flow Cytometry and mRNA Inhibition

Following siRNA treatment, cells were treated with compound no. 25690 or compound no. 395254 in the presence or absence of EGF. 16 hours after treatment with a PS-ASO with or without EGF, cells were harvested, and RNA levels were analyzed via RT-qPCR as in Example 8. Results are presented in the tables below. The results show that antisense activities of multiple PS-ASOs were decreased following inhibition of EGFR expression.

Uptake of compound no. 446654 was measured in siRNA treated A431 cells via flow cytometry, as described in example 8. The results are presented in the tables below and indicate that uptake of compound no. 446654 was unaffected by EGFR expression level.

TABLE 24
Antisense activity of PS-ASO complementary to Drosha

| [compound | Drosha mRNA (% Control) | | |
|---|---|---|---|
| no. 25690] | Control Luciferase | EGFR | EGFR |
| (nM) | siRNA | siRNA-1 | siRNA-2 |
| 97.7 | 100.0 | 100.0 | 100.0 |
| 195 | 81.4 | 103.0 | 88.0 |
| 391 | 67.0 | 78.9 | 83.1 |
| 781 | 56.6 | 62.3 | 64.8 |
| 1563 | 49.2 | 56.0 | 61.4 |
| 3125 | 40.7 | 51.4 | 54.6 |
| 6250 | 35.2 | 41.9 | 39.2 |
| 12500 | 29.3 | 32.4 | 39.1 |
| 25000 | 24.0 | 29.9 | 35.5 |
| 50000 | 22.4 | 25.7 | 31.2 |
| 100000 | 19.7 | 24.1 | 28.4 |
| 200000 | 14.9 | 18.5 | 20.8 |
| IC₅₀ (μM) | 1.31 ± 0.04 | 2.11 ± 0.10 | 2.79 ± 0.07 |

TABLE 25
Antisense activity of PS-ASO complementary to Malat1

| [compound | Malat1 RNA (% Control) | | |
|---|---|---|---|
| no. | Control | | |
| 395254] | Luciferase | EGFR | EGFR |
| (nM) | siRNA | siRNA-1 | siRNA-2 |
| 2.0 | 100.0 | 100.0 | 100.0 |
| 3.9 | 72.7 | 108.4 | 90.4 |
| 7.8 | 81.9 | 103.1 | 85.1 |
| 15.6 | 61.0 | 98.4 | 82.5 |
| 31.3 | 47.0 | 67.4 | 57.7 |
| 62.5 | 33.2 | 45.0 | 43.6 |
| 125 | 18.9 | 26.5 | 26.6 |
| 250 | 16.3 | 19.7 | 21.5 |
| 500 | 11.9 | 16.9 | 15.1 |
| 1000 | 7.9 | 11.3 | 12.7 |
| 2000 | 7.6 | 10.2 | 11.0 |
| 4000 | 1.3 | 7.8 | 11.1 |
| IC₅₀ (nM) | 46 ± 5.3 | 86 ± 9.3 | 100 ± 9.7 |

TABLE 26
Antisense activity of PS-ASO complementary to Drosha

| [compound | Drosha mRNA (% Control) | | | |
|---|---|---|---|---|
| no. | Control | Control | | EGFR |
| 25690] | Luciferase | luciferase + | EGFR | siRNA- |
| (nM) | siRNA | EGF | siRNA-2 | 2 + EGF |
| 97.7 | 100.0 | 100.0 | 100.0 | 100.0 |
| 195 | 99.2 | 110.2 | 105.2 | 107.2 |
| 391 | 100.0 | 100.0 | 100.0 | 100.0.0 |
| 781 | 77.5 | 60.8 | 89.8 | 98.4 |
| 1563 | 70.2 | 57.6 | 80.3 | 79.4 |
| 3125 | 61.6 | 40.8 | 70.6 | 75.4 |
| 6250 | 47.8 | 40.9 | 64.8 | 62.5 |
| 12500 | 41.0 | 30.9 | 49.2 | 47.1 |
| 25000 | 37.2 | 27.1 | 42.3 | 41.2 |
| 50000 | 33.1 | 23.2 | 29.3 | 31.3 |
| 100000 | 32.1 | 21.0 | 31.2 | 30.4 |
| 200000 | 25.2 | 21.4 | 26.1 | 25.1 |
| IC₅₀ (μM) | 1.5 ± 0.05 | 0.63 ± 0.04 | 2.73 ± 0.10 | 2.33 ± 0.11 |

TABLE 27
Antisense activity of PS-ASO complementary to Malat1

| [compound | Malat1 RNA (% Control) | | | |
|---|---|---|---|---|
| no. | Control | Control | | EGFR |
| 395254] | Luciferase | luciferase + | EGFR | siRNA- |
| (nM) | siRNA | EGF | siRNA-2 | 2 + EGF |
| 2.0 | 100 | 100 | 100 | 100 |
| 3.9 | 110.2 | 108.2 | 105.2 | 108.2 |
| 7.8 | 99.3 | 91.2 | 105.2 | 99.2 |
| 15.6 | 87.2 | 67.2 | 97.2 | 99.2 |
| 31.3 | 76.3 | 58.7 | 95.9 | 114.0 |
| 62.5 | 67.2 | 54.2 | 77.2 | 79.7 |
| 125 | 44 | 37.0 | 53.1 | 55.2 |
| 250 | 27.6 | 24.9 | 29.7 | 34.0 |
| 500 | 18.9 | 17.1 | 23.4 | 24.6 |
| 1000 | 18.2 | 15.2 | 17.2 | 21.4 |
| 2000 | 15.2 | 10.2 | 15.2 | 18.3 |
| 4000 | 11.2 | 7.2 | 15.2 | 17.2 |
| IC₅₀ (nM) | 49 ± 6.3 | 26 ± 2.4 | 89 ± 5.9 | 87 ± 4.2 |

TABLE 28
Uptake of Cy3-PS-ASO in siRNA-treated cells

| | RFU | | |
|---|---|---|---|
| | Control | | |
| Time | Luciferase | EGFR | EGFR |
| (hours) | siRNA | siRNA-1 | siRNA-2 |
| 0.25 | 1082 | 814 | 950 |
| 0.5 | 1988 | 1562 | 1637 |
| 1 | 2315 | 2308 | 2698 |
| 2 | 2276 | 2280 | 2349 |
| 4 | 3103 | 2854 | 3069 |
| 6 | 4300 | 3747 | 3821 |
| 8 | 4988 | 5048 | 5145 |

TABLE 29
Uptake of Cy3-PS-ASO in siRNA-treated cells

| [compound | RFU | | |
|---|---|---|---|
| no. | Control | | |
| 446654] | Luciferase | EGFR | EGFR |
| μM | siRNA | siRNA-1 | siRNA-2 |
| 0.015 | 22.3 | 25.3 | 24.7 |
| 0.03 | 31.3 | 33.0 | 29.0 |
| 0.06 | 136 | 133 | 101 |
| 0.12 | 359 | 444 | 292 |
| 0.25 | 806 | 879 | 852 |
| 0.5 | 1392 | 1437 | 1490 |
| 1.0 | 2374 | 2614 | 2906 |

### Example 11: Effects of EGFR Overexpression on Antisense Activity and Uptake

HEK cells were transfected with 2 μg plasmid encoding EGFR using Lipofectamine 3000 (ThermoFisher) at 2 μg/million cells. Cells were grown for 2 weeks in G418 selection media to select clones overexpressing EGFR. Cells were treated with compound no. 395254 for 16 hours prior to RT-qPCR analysis for Malat1, as in Example 8. The results, shown in the table below, indicate that antisense activity was increased in cells with higher expression levels of EGFR.

Uptake of compound no. 446654 was measured via flow cytometry, as in Example 8, in HEK cells overexpressing EGFR and wild type HEK cells. Results are presented in the table below. The results show that the varying EGFR expression levels did not affect PS-ASO uptake. Taken together, the results in several examples that EGFR mediated increased antisense activity but did not affect antisense oligonucleotide uptake indicates that EGFR mediated increased productive uptake.

TABLE 30
Antisense activity of PS-ASO complementary to Malat1

| [compound | Malat1 RNA (% control) | |
|---|---|---|
| no. | HEK cells | Wild type |
| 395254] | overexpressing | HEK |
| (nM) | EGFR | cells |
| 1 | 100 | 100 |
| 2 | 77.8 | 98.3 |
| 3.9 | 73.4 | 96 |
| 7.8 | 73.3 | 87.5 |
| 15.6 | 68.1 | 81.4 |
| 31.3 | 62.2 | 86.2 |
| 62.5 | 62.4 | 95.4 |
| 125 | 55.9 | 82.7 |
| 250 | 52.9 | 72.1 |
| 5000 | 44.1 | 54.8 |
| 1000 | 36.5 | 47.1 |
| 2000 | 35.3 | 28.5 |

TABLE 31
Uptake of Cy3-PS-ASO in HEK cells

| | RFU | |
|---|---|---|
| [compound | Wild | |
| no. | type | |
| 446652] | HEK | Overexpressing |
| (μM) | cells | EGFR |
| 0.25 | 109 | 123 |
| 0.5 | 143 | 163 |
| 1 | 287 | 255 |

1. A method comprising
contacting the cell with an EGFR modulator and
contacting a cell with an antisense compound comprising an antisense oligonucleotide, wherein the nucleobase sequence of the antisense oligonucleotide is complementary to a target nucleic acid.

2. The method of claim 1, wherein the expression of the target nucleic acid is reduced.

3. The method of claim 1 or 2, wherein the amount of the target nucleic acid is reduced.

4. The method of claim 1, wherein the target nucleic acid is a pre-mRNA, and the splicing of the target pre-mRNA is modulated.

5. The method of claim 1, wherein the expression of the target nucleic acid is increased.

6. The method of claim 1 or 5, wherein the amount of the target nucleic acid is increased.

7. The method of claim 2 or 3, wherein the expression or amount of the target nucleic acid is reduced to a greater extent than the extent of reduction of the expression or amount of the target nucleic acid that occurs in the absence of the EGFR modulator.

8. The method of claim 4, wherein the splicing of the target pre-mRNA is modulated to a greater extent than the extent of splicing modulation of the target pre-mRNA that occurs in the absence of the EGFR modulator.

9. The method of claim 5 or 6, wherein the expression or amount of the target nucleic acid is increased to a greater extent than the extent of increase of the expression or amount of the target nucleic acid that occurs in the absence of the EGFR modulator.

10. The method of any of claims 1-9, wherein the EGFR modulator is EGF.

11. The method of any of claims 1-9, wherein the EGFR modulator is TGF.

12. The method of claim 11, wherein the EGFR modulator is TGF alpha.

13. The method of any of claims 1-9, wherein the EGFR modulator is betacellulin.

14. The method of any of claims 1-9, wherein the EGFR modulator is heparin-binding EGF.

15. The method of any of claims 1-9, wherein the EGFR modulator is amphiregulin.

16. The method of any of claims 1-9, wherein the EGFR modulator is epigen.

17. The method of any of claims 1-9, wherein the EGFR modulator is epiregulin.

18. The method of any of claims 1-9, wherein the EGFR modulator is a second antisense compound comprising a second antisense oligonucleotide.

19. The method of claim 18, wherein the second antisense oligonucleotide is complementary to the 5′-UTR of EGFR.

20. The method of claim 18 or 19, wherein the second antisense oligonucleotide increases the expression of EGFR.

21. The method of any of claims 18-20, wherein the second antisense oligonucleotide is a modified oligonucleotide that is not a gapmer.

22. The method of claim 21, wherein the second antisense oligonucleotide is a fully modified oligonucleotide.

23. The method of any of claims 1-22, wherein the EGFR modulator modulates EGFR internalization.

24. The method of claim 23, wherein the EGFR modulator increases EGFR internalization.

25. The method of any of claims 1-22, wherein the EGFR modulator modulates EGFR signaling.

26. The method of any of claims 1-22, wherein the EGFR modulator modulates EGFR trafficking.

27. The method of any of claims 1-22, wherein the EGFR modulator modulates EGFR expression.

28. The method of claim 26, wherein the EGFR modulator increases EGFR expression.

29. The method of any of claims 1-27, wherein the antisense compound does not comprise the EGFR modulator.

30. The method of any of claims 1-28, wherein the EGFR modulator modulates wild type EGFR.

31. The method of claim 29, wherein the EGFR modulator does not modulate mutant EGFR.

32. The method of any of claims 1-29, wherein the EGFR modulator modulates mutant EGFR.

33. The method of any of claims 1-32, wherein the EGFR modulator increases productive uptake of the antisense compound.

34. The method of any of claims 1-33, wherein the nucleobase sequence of the antisense oligonucleotide is at least 80% complementary to the target nucleic acid.

35. The method of any of claims 1-33, wherein the nucleobase sequence of the antisense oligonucleotide is at least 85% complementary to the target nucleic acid.

36. The method of any of claims 1-33, wherein the nucleobase sequence of the antisense oligonucleotide is at least 90% complementary to the target nucleic acid.

37. The method of any of claims 1-33, wherein the nucleobase sequence of the antisense oligonucleotide is at least 95% complementary to the target nucleic acid.

38. The method of any of claims 1-33, wherein the nucleobase sequence of the antisense oligonucleotide is 100% complementary to the target nucleic acid.

39. The method of any of claims 1-38, wherein the antisense oligonucleotide is a modified oligonucleotide.

40. The method of claim 39, wherein the modified oligonucleotide is a gapmer.

41. The method of any of claims 1-41, wherein the antisense oligonucleotide comprises at least one modified internucleoside linkage.

42. The method of claim 41, wherein the at least one modified internucleoside linkage is a phosphorothioate internucleoside linkage.

43. The method of claim 41, wherein all of the internucleoside linkages of the antisense oligonucleotide are modified internucleoside linkages.

44. The method of claim 42, wherein all of the internucleoside linkages of the antisense oligonucleotide are phosphorothiate internucleoside linkages.

45. The method of claim 42, wherein all of the internucleoside linkages of the antisense oligonucleotide are selected from phosphorothioate and phosphate internucleoside linkages.

46. The method of any of claims 1-45, wherein the antisense compound is single-stranded.

47. The method of claim 46, wherein the antisense compound consists of a conjugate group and the antisense oligonucleotide.

48. The method of claim 46, wherein the antisense compound consists of the antisense oligonucleotide.

49. The method of any of claims 1-48, wherein the cell is in a population of rapidly proliferating cells.

50. The method of any of claims 1-49, wherein the cell is a cancer cell.

51. The method of any of claims 1-50, wherein the cell is a tumor cell.

52. The method of any of claims 1-51, wherein the cell is in an animal.

53. The method of claim 52, wherein the animal is a human individual.

54. The method of claim 53 comprising administering the EGFR modulator and the antisense compound to the individual.

55. The method of claim 54, wherein the individual has a disease or condition that is ameliorated or treated by the administration of the antisense compound.

56. The method of claim 55, wherein the disease or condition is cancer.

57. The method of any of claims 43-45, wherein the antisense compound and the EGFR modulator are administered simultaneously.

58. The method of any of claims 43-45, wherein the antisense compound and the EGFR modulator are administered sequentially.

59. Use of an antisense oligonucleotide having a nucleobase sequence complementary to a target nucleic acid in combination with an EGFR modulator.
